(19) United States
(12) Patent Application Publication
(10) Pub. No.: US 2026/0000555 A1
(43) Pub. Date: Jan. 1, 2026

(54) RECEIVING LAYER FOR AN ABSORBENT ARTICLE

(71) Applicant: The Procter & Gamble Company, Cincinnati, OH (US)

(72) Inventor: Gerard Alain VIENS, Wyoming, OH (US)

(21) Appl. No.: 19/246,862

(22) Filed: Jun. 24, 2025

Related U.S. Application Data
(60) Provisional application No. 63/664,376, filed on Jun. 26, 2024.

(51) Int. Cl. A61F 13/512; A61F 13/511

(52) U.S. Cl. CPC A61F 13/512; A61F 13/5116; A61F 2013/51178

(57) ABSTRACT

A receiving layer having a basis weight of from about 40 gsm to about 75 gsm and including a first sublayer and a second sublayer. Each of the first sublayer and the second sublayer are a nonwoven comprising fibers having a decitex less than about 2. The second sublayer comprises from about 15 to about 35 weight percent of cellulosic fibers, from about 65 to about 85 weight percent of bonding fibers. The receiving layer has a caliper factor of from about 0.26 mm to about 0.35 mm. The receiving layer can be included in a disposable absorbent article.

### CROSS REFERENCE TO RELATED APPLICATIONS

This application claims the benefit, under 35 U.S.C. § 119 (e), to U.S. Provisional Application No. 63/664,376, filed Jun. 26, 2024, the entire disclosure of which is fully incorporated by reference herein.

### FIELD OF THE INVENTION

The present disclosure generally relates to a receiving layer for a disposable absorbent article, in particular, a receiving layer that is a needle punched nonwoven layer having improved performance characteristics.

### BACKGROUND

Disposable absorbent articles such as feminine hygiene products, taped diapers, pant-type diapers, and incontinence products are designed to absorb fluids from the wearer's body. Users of such disposable absorbent articles have several concerns. For example, leakage from products like catamenial pads, diapers, sanitary napkins, and incontinence pads is a significant concern.

Additionally, comfort and the feel of the product against the wearer's body is also a concern. To provide better comfort, current disposable absorbent articles are typically provided with a topsheet that is flexible, soft feeling, and non-irritating to the wearer's skin. The topsheet does not itself hold the discharged fluid. Instead, the topsheet is fluid-permeable to allow the fluids to flow into an absorbent core. The fluid management layer is used to facilitate movement of fluid from the topsheet to the absorbent core. The fluid management layer is key to maintaining a dry feeling absorbent article that inhibits leakage.

Regarding comfort, some consumers may desire a product that has sufficient thickness and provides the desirable amount of protection while also being flexible and relatively cushy. Lofty materials may be utilized to provide a thick cushiony feeling article. However, in use these lofty materials can experience various compressive loads. Recovery from these compressive loads is paramount in maintaining the cushiony feeling of the article. Exacerbating this issue is the fact that the characteristics of the materials of the absorbent article change once fluid is introduced into the article. Hence, an article that may meet a consumer's requisite criteria before use may no longer be comfortable, flexible, or have the desired stiffness to the user after a given amount of fluid has been absorbed by the absorbent article.

Traditionally, absorbent articles include a separate and distinct topsheet, fluid management layer, and absorbent core. These layers are then bonded together with an adhesive. To manufacture these separate layers is relatively costly and request additional logistical considerations in getting materials to the converting manufacturing line to assemble the absorbent article. Further, the use of adhesives between each of these layers adds cost and complexity to the manufacturing process. Also, there is a group of consumers that would prefer to minimize the amount of adhesives in their absorbent articles. Further still, because these layers are separate and joined by adhesives, these layers have relatively large separation or gaps between the layers that allow fluid to accumulate in places, which leads to increased leakage and rewet.

As such there is a need to create a layer that has sufficient caliper and consumer desired recovery properties for use in absorbent articles while still delivering sufficient capillary action to facilitate fluid moving to the absorbent core while minimizing leakage and rewet.

### SUMMARY OF THE INVENTION

In some embodiments, an absorbent article may comprise: a receiving layer, a backsheet, and an absorbent core disposed between the receiving layer and the backsheet. The receiving layer may comprise a multi-sublayer structure, and the multi-sublayer structure may comprise a first sublayer and a second sublayer. The first sublayer is disposed adjacent to a wearer's skin and has a basis weight of from about 15 gsm to about 45 gsm. The second sublayer is located below the first sublayer and between the first sublayer and the absorbent core. The second sublayer comprises nonwoven fibers having a basis weight of from about 40 gsm to about 75 gsm. The nonwoven fibers comprising from about 15 to about 35 weight percent of cellulosic fibers, from about 65 to about 85 weight percent of bonding fibers, and the cellulosic fibers and the bonding fibers have a decitex of less than about 2. The first sublayer is integrated with the second sublayer, and the integration comprises micro-fiber integration and large-fiber integration.

In some embodiments, an absorbent article may comprise: a receiving layer, a backsheet, and an absorbent core disposed between the receiving layer and the backsheet. The receiving layer comprises a multi-sublayer structure comprising a first sublayer and a second sublayer. The first sublayer is disposed adjacent to a wearer's skin, and the first sublayer has a basis weight of from about 15 gsm to about 45 gsm. The second sublayer is located below the first sublayer and between the first sublayer and the absorbent core. The second sublayer comprises nonwoven fibers having a basis weight of from about 40 gsm to about 75 gsm. The nonwoven fibers comprising from about 15 to about 35 weight percent of cellulosic fibers, from about 65 to about 85 weight percent of bonding fibers, and the cellulosic fibers and the bonding fibers have a decitex of less than about 2. The first sublayer is integrated with the second sublayer, and the integration comprises a plurality of large-fiber integration areas. Additionally, the first sublayer has a first mean pore size, and the second sublayer has a second mean pore size, and the second mean pore size is at least about 40% of the first mean pore size, as determined by the Micro-CT Pore Size Measurement Method.

In some embodiments, an absorbent article may comprise: a receiving layer, a backsheet, and an absorbent core disposed between the receiving layer and the backsheet, and the receiving layer comprising a multi-sublayer structure. The multi-sublayer structure may comprise a first sublayer and a second sublayer. The first sublayer is disposed adjacent to a wearer's skin, and the first sublayer has a basis weight of from about 15 gsm to about 45 gsm. The second sublayer is located below the first sublayer and between the first sublayer and the absorbent core. The second sublayer comprises nonwoven fibers having a basis weight of from about 40 gsm to about 75 gsm. The nonwoven fibers comprising from about 15 to about 35 weight percent of cellulosic fibers, from about 65 to about 85 weight percent of bonding fibers, and the cellulosic fibers and the bonding fibers have a decitex of less than about 2. The first sublayer is integrated with the second sublayer, and the integration comprises a plurality of large-fiber integration areas. The first sublayer has a first through plane permeability and the second sublayer has a second through plane permeability, and the second through plane permeability is at least about 30% of the first through plane permeability.

### BRIEF DESCRIPTION OF THE DRAWINGS

FIG. 1 is a schematic representation of a disposable absorbent article.

FIG. 1A is a schematic representation of a disposable absorbent article.

FIG. 2 is a schematic representation of a disposable absorbent article.

FIG. 3 is a schematic representation of a disposable absorbent article constructed in accordance with the present disclosure.

FIG. 4 is a schematic representation of a disposable absorbent article constructed in accordance with the present disclosure.

FIG. 4B is a cross-sectional view taken along line 4B-4B of the disposable absorbent article of FIG. 4.

FIG. 5A is an image of cross-sectional view of a receiving layer and a schematic representation of the fibers of the receiving layer.

FIG. 5B is an image of cross-sectional view of a topsheet disposed on a fluid management layer and a gap therebetween and a schematic representation of the fibers of the topsheet and the fluid management layer and a gap therebetween.

FIG. 6 is a schematic representation of a manufacturing process to form a receiving layer web.

FIG. 6A is top view of a receiving layer (no cross lapping for comparison).

FIG. 6B is a top view of a receiving layer with needlepunch.

FIG. 7A is a side view of a receiving layer with a needlepunch cross section with a vertical fiber bundle.

FIG. 7B is a side view of a receiving layer with needlepunch cross section with multiple vertical fiber bundles.

FIG. 7C is a top view of a view a receiving layer with needlepunch cross section with multiple engagement areas from the plurality of needles forming vertical fiber bundles.

FIG. 7D is a top view of a receiving layer top view with areas of capillary boosting points.

FIG. 8A is a schematic cross section of a measurement apparatus configuration used in the Permeability Measurement Method described herein, taken through a vertical plane that bisects the depicted fluid vessel.

FIG. 8B is a view of the measurement apparatus as illustrated in FIG. 8A, illustrated with added elements in preparation for commencement of a measurement procedure.

FIG. 8C is a view of the measurement apparatus as illustrated in FIG. 8B, illustrated following commencement of a measurement procedure.

FIG. 9A is a perspective view of a sample weight used in the Permeability Measurement Method described herein.

FIG. 9B is a top view of the sample weight depicted in FIG. 9A.

FIG. 9C is a vertical cross section view of the sample weight depicted in FIG. 9A.

FIG. 10 is a top view of a sample support used in the Permeability Measurement Method described herein.

### DETAILED DESCRIPTION

### Definitions

As used herein, the following terms shall have the meaning specified thereafter:

“Absorbent article” refers to wearable devices, which absorb and/or contain liquid, and more specifically, refers to devices, which are placed against or in proximity to the body of the wearer to absorb and contain the various exudates discharged from the body. Absorbent articles can include diapers, training pants, adult incontinence undergarments (e.g., liners, pads and briefs) and/or feminine hygiene products, including feminine hygiene pads (also known as, for example, “sanitary napkins”, “menstrual pads”, “panty liners”, etc.).

The term “integrated” as used herein is used to describe fibers of a nonwoven material which have been intertwined, entangled, and/or pushed/pulled in a positive and/or negative Z-direction (direction of the thickness of the nonwoven material). Some exemplary processes for integrating fibers of a nonwoven web include spunlacing and needlepunching. Spunlacing (also known as “hydroentangling” or (“hydro-enhancing”) uses a plurality of high pressure water jets directed at a precursor batt or accumulation of fibers being conveyed along a machine direction, to entangle the fibers. Needlepunching (also known as “needling”) involves the use of specially-featured needles to mechanically push and/or pull fibers, of a precursor batt or accumulation of fibers, in a z-direction, to entangle them with other fibers in the batt or accumulation.

The term “carded” as used herein is used to describe structural features of the receiving layer described herein. A carded nonwoven web is formed of fibers which are cut to a specific finite length, otherwise known as “staple length fibers.” Staple length fibers may be of any selected length. For example, staple length fibers may be cut to a length of up to 120 mm, to a length as short as 10 mm. However, if fibers of a particular group are staple length fibers, then the length of each of the fibers in the carded nonwoven is approximately the same, i.e., the staple length. Where fibers of more than one composition are included in a nonwoven web, for example, a web including polypropylene fibers and viscose fibers, the length of each fiber of the same composition may be substantially the same, while the respective staple fiber lengths of the respective fiber compositions may differ.

In contrast to staple fibers, filaments such as those produced by spinning, e.g., in a spunbond or meltblown nonwoven web manufacturing processes, are not ordinarily staple length fibers. Instead, these filaments are sometimes characterized as “continuous” fibers, meaning that they are of a relatively long and indeterminate length, not cut to a specific length following spinning, as their staple fiber counterparts are.

The term “lateral” as used herein with respect to an absorbent article such as a feminine hygiene pad, or a component thereof, refers to a direction parallel to a horizontal line tangent to the front surfaces of the upper portions of wearer's legs proximate the torso, when the pad is being worn normally and the wearer has assumed an even, square, normal standing position. A “width” dimension of any component or feature of an article such as a feminine hygiene pad is measured along the lateral direction. When the article or component thereof is laid out flat on a horizontal surface, the “lateral” direction corresponds with the lateral direction relative the structure when it is worn, as defined above. With respect to an article such as a feminine hygiene pad that is opened and laid out flat on a horizontal planar surface, “lateral” refers to a direction perpendicular to the longitudinal direction and parallel to the horizontal planar surface.

The “lateral axis” of an absorbent article such as a feminine hygiene pad or component thereof is a lateral line lying in an x-y plane and equally dividing the length of the pad or the component when it is laid out flat on a horizontal surface. A lateral axis is perpendicular to a longitudinal axis.

The term “longitudinal” as used herein with respect to an absorbent article such as a feminine hygiene pad, or a component thereof, refers to a direction perpendicular to the lateral direction. A “length” dimension of any component or feature of the article is measured along the longitudinal direction from its forward extent to its rearward extent. When an article such as a feminine hygiene pad or component thereof is laid out flat on a horizontal surface, the “longitudinal” direction is perpendicular to the lateral direction relative the pad when it is worn, as defined above.

The “longitudinal axis” of a feminine hygiene pad or component thereof is a longitudinal line lying in an x-y plane and equally dividing the width of the pad or component, when the pad is laid out flat on a horizontal surface. A longitudinal axis is perpendicular to a lateral axis.

The term “x-y plane,” with reference to an absorbent article, such as a feminine hygiene pad, or component thereof, when laid out flat on a horizontal surface, means any horizontal plane occupied by the horizontal surface or any layer of the article or component.

The term “z-direction,” with reference to an absorbent article, such as a feminine hygiene pad or component thereof, when laid out flat on a horizontal surface, is a direction perpendicular/orthogonal to the x-y plane.

The terms “top,” “bottom,” “upper,” “lower,” “over,” “under,” “beneath,” “superadjacent,” “subjacent,” and similar terms relating to relative vertical positioning, when used herein to refer to layers, components or other features of an absorbent article such as a feminine hygiene pad, are relative the z-direction and are to be interpreted with respect to the pad as it would appear when laid out flat on a horizontal surface, with its wearer-facing surface oriented upward and outward-facing surface oriented downward.

With respect to an absorbent article such as a feminine hygiene pad, or a component or structure thereof, “wearer-facing” is a relative locational term referring to a feature of the component or structure that when in use that lies closer to the wearer than another feature of the component or structure. For example, a receiving layer has a wearer-facing surface that lies closer to the wearer than the opposite, outward-facing surface of the receiving layer.

With respect to an absorbent article such as a feminine hygiene pad, or a component or structure thereof, “outward-facing” is a relative locational term referring to a feature of the component or structure that when in use that lies farther from the wearer than another feature of the component or structure. For example, a receiving layer has an outward-facing surface that lies farther from the wearer than the opposite, wearer-facing surface of the receiving layer.

“Machine Direction” or “MD” as used herein with respect to an absorbent article such as a feminine hygiene pad or component thereof, refers to a direction parallel to the flow of the article or component through processing/manufacturing equipment.

“Cross Machine Direction” or “CD” as used herein with respect to an absorbent article such as a feminine hygiene pad or component thereof, refers to a direction perpendicular/orthogonal to the machine direction.

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

### Feminine Hygiene Pad

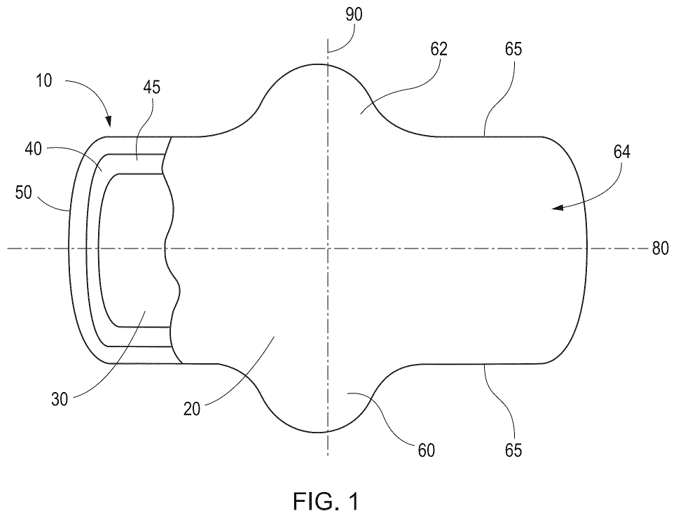

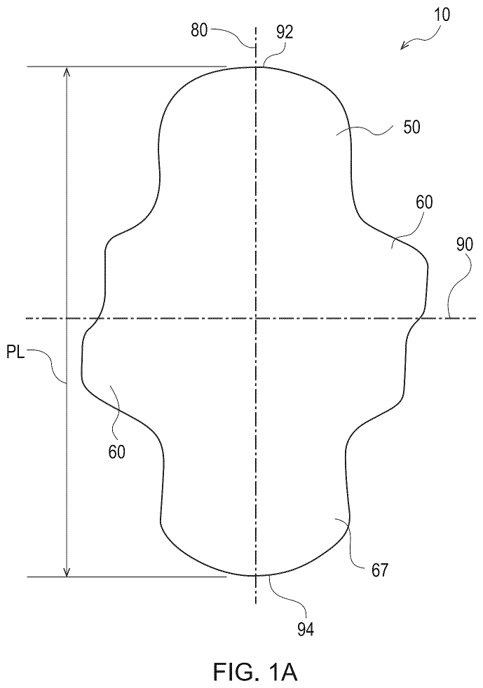

Referring to FIG. 1, an absorbent article as contemplated herein, such as a feminine hygiene pad 10, will include a wearer-facing surface and an opposing outward-facing surface. A liquid permeable topsheet 20 may form at least a portion of the wearer-facing surface and a liquid impermeable backsheet may form at least a portion of the outward-facing surface. An absorbent core including an absorbent structure 40 is disposed between the topsheet and the backsheet, and a fluid management layer 30 may be included and disposed between the absorbent structure 40 and the topsheet 20. (A fluid management layer as described herein is sometimes known in the art as an “acquisition/distribution layer”, “distribution layer”, or “secondary topsheet”, whose purpose is to dissipate energy from a fluid gush to the extent needed, provide a temporary volume of space for discharged fluid to occupy during the time required for an underlying absorbent structure to imbibe and absorb the fluid, and to distribute the fluid across the absorbent structure to maximize effective use thereof.) Non-limiting examples of absorbent articles sharing these features include feminine hygiene pads (also known as “sanitary napkins”, “menstrual pads,” etc.), disposable incontinence pads, disposable incontinence underwear, disposable baby diapers and disposable baby/child training pants.

The topsheet 20 and the backsheet 50 may be joined together to form and define an outer periphery 65 of the pad 10. The absorbent structure 40, including an absorbent core 45, and the fluid management layer 30 may each be sized to have outer perimeters disposed laterally and longitudinally inboard of the outer periphery 65. The absorbent structure 40 and the fluid management layer 30 may be dimensioned and shaped substantially similarly or identically to each other in the x-y directions, or they may have respective differing x-y dimensions and/or shapes. One or both may be manufactured to have a rectangular shape as suggested in FIG. 1, or one or both may be manufactured to have any other suitable shape, such as an oval shape, stadium shape, rounded rectangle shape, hourglass shape, peanut shape, etc. Shapes having concave profiles along the longitudinal edges may in some examples provide for enhanced comfort and/or conformity with the wearer's body.

The topsheet 20 may be joined to the backsheet 50 by any suitable attachment mechanism.

The topsheet 20 and the backsheet 50 may be joined directly to each other in the article periphery 65, and may be indirectly joined together by directly joining them to the absorbent structure 40, the fluid management layer 30, and/or additional layers disposed between the topsheet 20 and the backsheet 50. This indirect or direct joining may be accomplished by any suitable attachment mechanism known in the art. Non-limiting examples of attachment mechanisms may include e.g., fusion bonds, ultrasonic bonds, pressure bonds, adhesive bonds, or any suitable combinations thereof. The absorbent article 10 may also comprise wings 60 extending outwardly with respect to a longitudinal axis 80 of the absorbent article 10. As illustrated in FIG. 1A, the wings may be asymmetric, such as disclosed, for example, in U.S. patent publication numbers 2022/0409449, 2021/0307977, 2018/0325750, and 2018/0325751, which are all incorporated herein by reference. The wings may be asymmetric about at least one of the longitudinal axis 80 and the lateral axis 90. The pad may have a pad length PL taken parallel to longitudinal axis from the first lateral edge 92 to the second lateral edge 94. The wings 60 may be positioned in the central region of the absorbent article, such as illustrated in FIG. 1A.

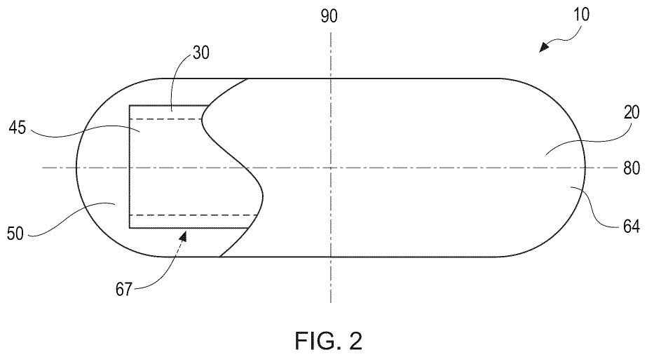

In some embodiments, referring to FIG. 2, the disposable absorbent article 10 having a topsheet 20, a backsheet 50, an absorbent core 45, disposed between the topsheet and the backsheet, and a fluid management layer 30 disposed between the topsheet and the absorbent core. As illustrated in FIG. 2, the absorbent article 10 may not have a wing. Further, the absorbent core 45 and the fluid management layer 30 may be sized such that a portion of the absorbent core 45 extends beyond one or more sides of the fluid management layer 30 and/or a portion of the fluid management layer 30 extends beyond one or more side edges of the absorbent core 45. The topsheet 20 may form the wearer-facing surface of the pad, such that the topsheet is the first layer to receive any bodily exudates. The fluid management layer 30 may disposed adjacent the topsheet 20 and be positioned between the topsheet 20 and the absorbent core 45. The fluid management layer 30 aid in wicking the bodily fluid away from the topsheet quickly and transferring the fluid to the absorbent core. The topsheet and the fluid management layer work together to quickly absorb and transfer fluid. As previously discussed, the absorbent core 45 holds to the bodily fluid and the backsheet 50 protects the wearer's undergarments by not allowing fluid to pass through the backsheet layer. The backsheet provides the garment facing layer of the absorbent article.

As described, there are two layers, the topsheet layer and the fluid management, layer that work together to absorb and wick fluid. Traditionally, these two layers are manufactured separately and the topsheet web and the fluid management web are stored on rolls that are to be used on a converting manufacturing line. The rolls are supplied to the converting manufacturing line where the two separate materials are unwound, processed, and assembled into an absorbent article. The topsheet layer and the fluid management layer are typically bonded to one another by adhesive or other bonding at specific bond sites. Because these layers are separate and then combined, these layers often have gaps outside of the bond sides or adhesive areas where fluid can get trapped. This trapped fluid is at risk of leaking out of the pad and making the pad feel relatively more wet because it can transfer back to the topsheet or out of the edges of the pad. Eliminating or minimizing these gaps between the fluid management layer and the topsheet would aid in reducing leakage and minimizing rewet of the absorbent article.

Further, the need for two separate layers of material that are separately manufactured adds to the cost of manufacturing and the complexity in supply chain to transport and have on hand the required amount of materials. Thus, it would be beneficial to produce a layer on the same manufacturing line that retains the properties of the topsheet layer and the fluid management layer.

The following disclosure describes the receiving layer which is a layer comprising a multi-sublayer structure. The receiving layer may comprise a first sublayer and a second sublayer. The gaps between the first sublayer and the second sublayer are eliminated or minimized, which will 20) be descried in more detail herein. Further, the receiving layer is manufactured such that each of the first sublayer and the second sublayer are manufactured as a single web of material that can then be wound and supplied to the converting manufacturing line where the absorbent article is manufactured, which will be described in more detail herein. Additionally, there is no adhesive between the first sublayer and the second sublayer. Stated another way, the receiving layer is free of adhesive and gaps between the first sublayer and the second sublayer.

### Receiving Layer

The receiving layer 101 is a multi-sublayer structure comprising at least two sublayers, such as a first sublayer and a second sublayer, but may comprise greater than 2 layers. For example, the receiving layer may include three sublayers or more layers up to six layers. As previously discussed, it is desirable that the receiving layer provide high dryness/low rewet and rapid acquisition or, stated another way, rapid movement of fluid away from the wearer's skin, and be soft feeling, and non-irritating to the wearer's skin.

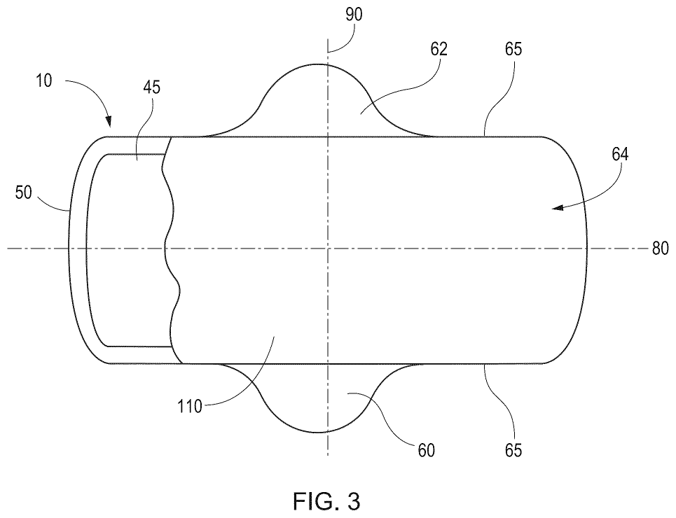

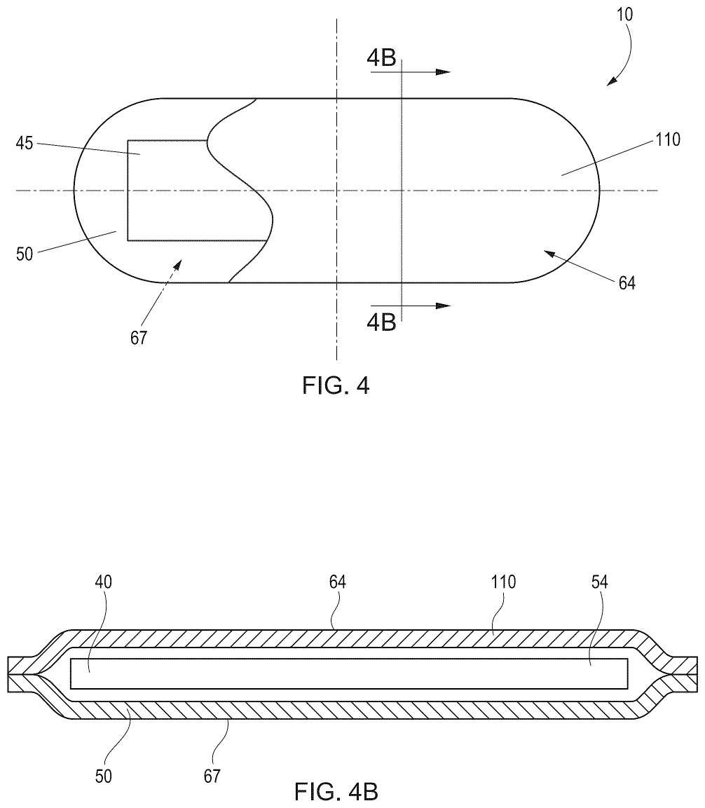

As illustrated in FIGS. 3, 4, and 4A, the receiving layer 110 is forms at least a portion of the wearer-facing surface 64 of the absorbent article 10. The receiving layer 110 may be disposed on the absorbent core 45 or absorbent structure 40. The receiving layer 110 may be joined to a portion of the backsheet 50 to seal the absorbent core 45 therebetween. At least one of the receiving layer 110 and the backsheet 50 defines at least a portion of the outer periphery 65. The size of the receiving layer 110 may be the same as or different than the size of the backsheet 50. Further, as illustrated in FIG. 3, the absorbent article may include a wing 60. The wing 60 may extend from a portion of the receiving layer 110. The wing 60 may extend away from a first longitudinal edge of the receiving layer. Similarly, a second wing may extend away from a second longitudinal edge of the receiving layer. The wing 60 may be joined to at least one of the receiving layer 110 and the backsheet 50. The wing 60 may be a separate material joined to the receiving layer 110 or the backsheet 50, or the wing 60 may be formed from at least one of the backsheet 50 and the receiving layer 110. As previously discussed, the wings may be asymmetric with respect to at least one of the longitudinal axis and the lateral axis. The absorbent structure 40 and the receiving layer 110 may be dimensioned and shaped substantially similar or identically to each other in the x-y directions, or they may have respective differing x-y dimensions and/or shapes. One or both may be manufactured to have any other suitable shape, such as a rectangle shape, an oval shape, stadium shape, rounded rectangle shape, hourglass shape, peanut shape, etc. Shapes having concave profiles along the longitudinal edges may in some examples provide for enhanced comfort and/or conformity with the wearer's body.

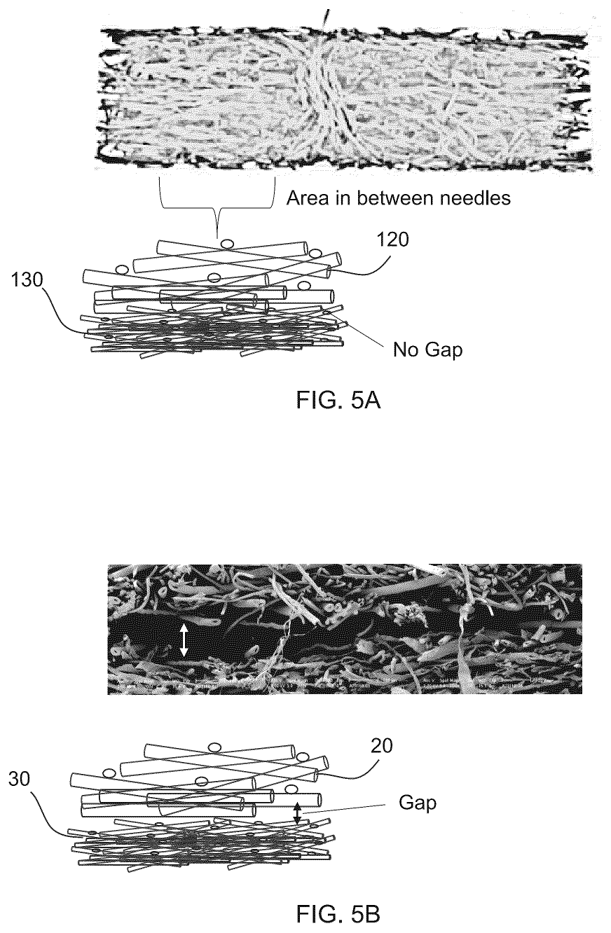

As previously discussed, the receiving layer 110 comprises at least a first sublayer 120 and a second sublayer 130. The first sublayer 120 and the second sublayer 130 are manufactured together on a single manufacturing line, which allows the fibers to have micro-fiber integration. Micro-fiber integration refers to the fibers of each of the first sublayer 120 and the second sublayer 130 integrating with one another such that a portion of the fibers of the first sublayer 120 overlap and/or enmesh with a portion of the fibers of the second sublayer 130, which eliminates the gap between the first sublayer and the second sublayer, as illustrated in FIG. 5A for example. More specifically, the first sublayer 120 is disposed on the second sublayer 130 in the manufacturing process when the fibers are relatively loose or prior to any bonding or attachment between the fibers. Allowing the first substrate to be laid on the second substrate prior to any bonding or attachment between the fibers, allows the fibers to move with respect to one another proving the fibers the ability to integrate with one another, eliminating and/or minimizing gaps, and forming micro-fiber integration between the fibers. A gap is a separation between the first layer and the second layer. A gap is a separation between two adjacent layers having a gap height of at least 80 microns and a gap width of at least 100 microns, according to the Gap SEM Imaging Method disclosed herein. The receiving layer 110, due to the type of fibers (decitex less than about 2) and the manufacturing process, includes no gaps (is free of gaps) or minimizes the gaps such that any gap that is present has a gap height less than 80 microns and a gap width of less than 100 microns, according to the Gap SEM Imaging Method.

In addition to the micro-fiber integration, the first sublayer 120 and the second sublayer 130 are macro-integrated by needlepunching or needling. By integrally forming the first and second layers together on the same nonwoven manufacturing line, as shown in the present invention, superior performance in rapid acquisition and dryness can be achieved beyond previous absorbent articles. By contrast, as illustrated in FIG. 5B, traditionally, the topsheet 20 and the fluid management layer 30 are separately manufactured layers that are then placed in a layered configuration and combine by adhesive and/or thermal bonding. This separate manufacturing and post bonding of the two layers leads to gaps between the material layers which allows for accumulation of fluid within the gap and a higher likelihood of rewet and failure for the fluid to be absorbed and trapped by the core leading to leakage. By accumulating fibers, carding, and needlepunching the combined first and second layers within a singular manufacturing line both micro-fiber integration and large-fiber integration is possible without the presence of structural gaps between the layers. It is to be appreciated that other mechanical integrating means may be used to macro-integrate the first sublayer 120 and the second sublayer 130, such as a roll including a plurality of pins extending from the roll surface that are used to penetrate and integrate the sublayers and other similar mechanically penetrating devices.

The receiving layer as contemplated herein may be manufactured to have a combined basis weight of about 40 gsm to 120 gsm, more preferably about 65 gsm to 100 gsm, or most preferably about 65 gsm to 95 gsm, specifically reciting all values within these ranges and any ranges created thereby. The receiving layer 110 has a caliper. The caliper of the receiving layer may be from about 0.95 mm to about 1.5 mm at 2 kPa. It is to be appreciated that the caliper of the receiving layer may vary from about 5% to about 30% depending on when the caliper is measured. For example, the caliper of the receiving layer web, as will be described herein, may have a higher caliper than the receiving layer in the finished product. The caliper of the receiving layer web prior to winding may be greater than the caliper of the receiving layer in the finished product, such as an absorbent article because the receiving layer web undergoes winding which may compress the layer and subsequent processing prior to being incorporated into the product. Thus, the range of calipers for the receiving layer may vary. The caliper may be evaluated when the receiving layer 110 web advances from the receiving layer manufacturing line and prior to first winding the receiving layer 110 web. Additionally, the caliper of the receiving layer 110 may be evaluated in the finished product. The caliper of the receiving layer 110 is dependent on the caliper of the first sublayer and the second sublayer, which will be discussed in more detail herein. The first sublayer may have a caliper of from about 0.20 mm to about 0.3 mm or from about 0.22 mm to about 0.25 mm at 2 kPa. The second sublayer may have a caliper of from about 0.7 mm to about 1.2 mm at 2 kPa. Similarly, these caliper ranges may vary depending on when the caliper of the sublayer is measured. The caliper is determined according to the caliper test method disclosed herein.

To properly handle fluid, the properties of the first sublayer 120 must be balanced with the properties of the second sublayer 130. Generally, the first sublayer 120 has a first mean pore size and the second sublayer 130 has a second mean pore size. The first mean pore size is greater than the second mean pore size so that fluid it acquired more quickly and transfers more rapidly to the second sublayer 130. The second mean pore size may be from about 40% to about 90% or from about 50% to about 85% or from about 60% to about 80% of the first mean pore size. Additionally, the through plane permeability of the first sublayer 120 should be selected in view of the through plane permeability of the second sublayer 130. The first sublayer 120 has a first through plane permeability and the second sublayer 130 has a second through plan permeability. The second through plane permeability is at least about 30% of the first through plane permeability. The second through plane permeability may be from about 30% to about 70% or from about 35% to about 65% or from about 40% to about 60% of the first through plane permeability. The mean pore size is determined by the Micro-CT Pore Size Measurement Method disclosed herein. The through plane permeability is determine by the Permeability Measurement Method disclosed herein.

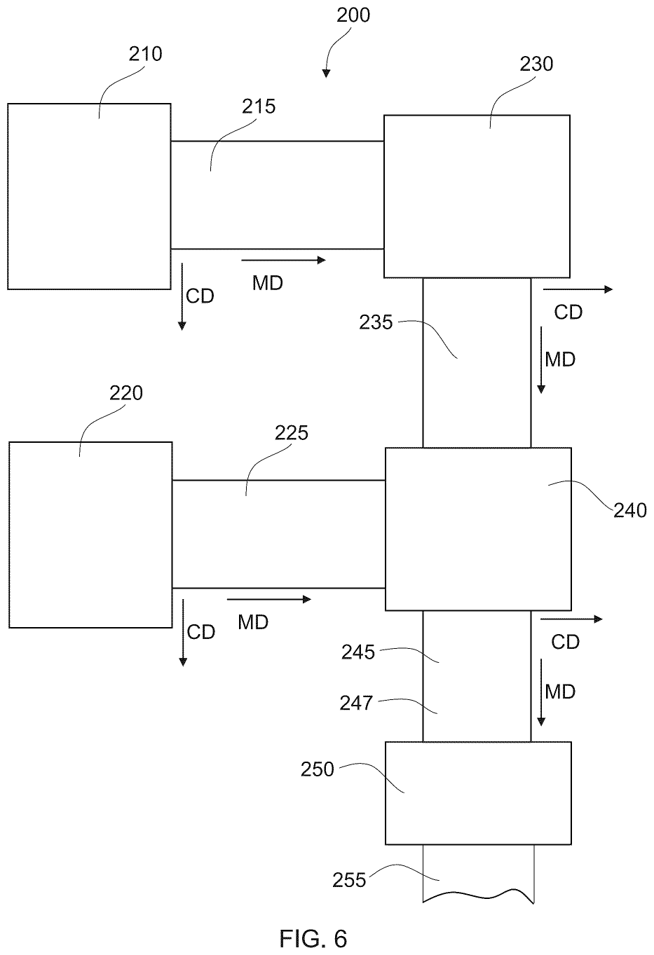

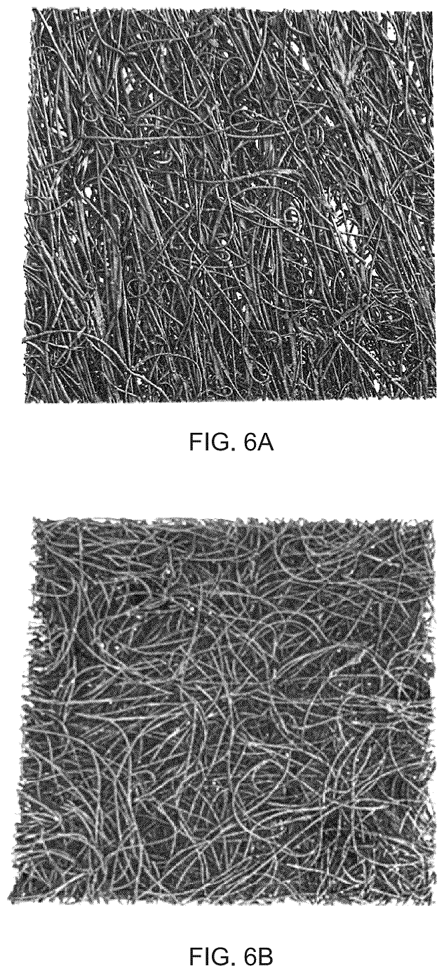

To manufacture this inventive receiving layer 110, it has been found that the sublayers be, preferably, crosslapped and integrated via the use of specially crafted needles through a needle punching process. This delivers the desired MD and CD material tensiles (reported as peak load) and MD/CD tensile ratios and maintains the overall caliper as the energy to entangle is concentrated to specific localized areas in the web rather than the entire web or substantial sections of the web, such as when adhesive is used. FIG. 6 is a schematic representation of the web manufacturing process 200. The web manufacturing process 200 comprises a first carding device 210 and a second carding device 220. Carding is a mechanical process using staple fibers. To obtain staple fibers, the fibers are first spun, cut to the required length, and put into bales (bundles of compressed fibers). The carding process starts with the opening of the bales of fibers. The fibers are then combed into a web by a carding device or machine, such as a rotating drum or series of drums covered in fine wires or teeth. The precise configuration of cards will depend on the fabric weight and fiber orientation required. The web can be parallel-laid, where most of the fibers are laid in the direction of the web travel, or they can be random-laid. Typical parallel-laid carded webs result in good tensile strength, low elongation, and low tear strength in the machine direction and the reverse in the cross direction, such as disclosed in U.S. Patent Publication No. 2022/0133553. It is also to be appreciated that crosslapping each of the first sublayer and the second sublayer is preferred to obtain the desired fluid handling properties and to maintain the desired caliper of the layer, but other configurations of forming the webs may be possible by altering the properties of the fibers to still obtain the desired fluid handling properties of the layer.

The nonwoven of the present invention is initially fiber blended, accumulated and laydown and fed through one or more carding steps. The initial carded web 215 of the first sublayer 120 may be produced by the first carding device 210. The initial carded web 215 of the first sublayer 120 is laid down onto a conveyor, belt, or some moveable support because the fibers are not at all integrated at this point and do not have structural integrity to be advanced without the support of a conveyor. The initial carded web 215 of the first sublayer 120 is advanced in a machine direction MD to a first cross-lapping device 230. The first cross-lapping device 230 layers the initial carded web of the first sublayer 120 to form a layered carded web 235 of the first sublayer. The number of layers will depend on the needed caliper and process parameters of the final absorbent article. Similarly, the initial carded web 225 of the second sublayer 130 may be produced by the second carding device 220. The initial carded web 225 of the second sublayer 130 is laid down onto a conveyor, belt, or some moveable support because the fibers are not at all integrated at this point and do not have structural integrity to be advanced without the support of a conveyor. The initial carded web 225 of the second sublayer 130 is advanced in a machine direction MD to a second cross-lapping device 240.

The non-woven, initial carded web 225 material is then crosslapped prior to the web forming process step. Crosslapping is well known to a person skilled in the art. For example, the carded web material is moved forward and backwards when laid on a belt or carrier while its lower front portion is pulled perpendicular to this forward and backward movement whereby the web material overlaps in a z-like fashion. This imparts sufficient MD/CD tensile ratio and builds the desired basis weight. The first cross-lapping device 240 layers the initial carded web 225 of the first sublayer 120 to form a layered carded web 245 of the second sublayer 130. As previously stated, the number of layers will depend on the needed caliper and process parameters of the final absorbent article. FIG. 6A illustrates an example of a portion of a web that is not cross-lapped. FIG. 6B is an example of a portion of a web that has undergone cross-lapping, which shows the fibers are oriented in both the machine direction MD and cross machine direction CD. Either downstream of the second cross-lapping device 240 or within the second cross-lapping device 240, the layered carded web 245 of the second sublayer is disposed on the layered carded web 235 of the first sublayer forming a precursor receiving web 247. It is to be appreciated that the layered carded web 245 of the second sublayer 130 and the layered carded web 235 of the first sublayer 120 have not been integrated and therefore include fibers that are moveable. Thus, the fibers of the layered carded web 245 of the second sublayer 130 and the fibers of the layered carded web 235 of the first sublayer 120 may move with respect to one another and accumulate in any open areas forming micro-fiber integration between the two sublayers.

It is also to be appreciated that the layers may be manufactured and laid down in either order. For example, the second sublayer layer 130 may be manufactured by the first carding device 210 and the first cross-lapping device and the first sublayer may be manufactured by the second carding device 220 and the second cross-lapping device 240. The order of manufacture may be based on the final configuration of the web.

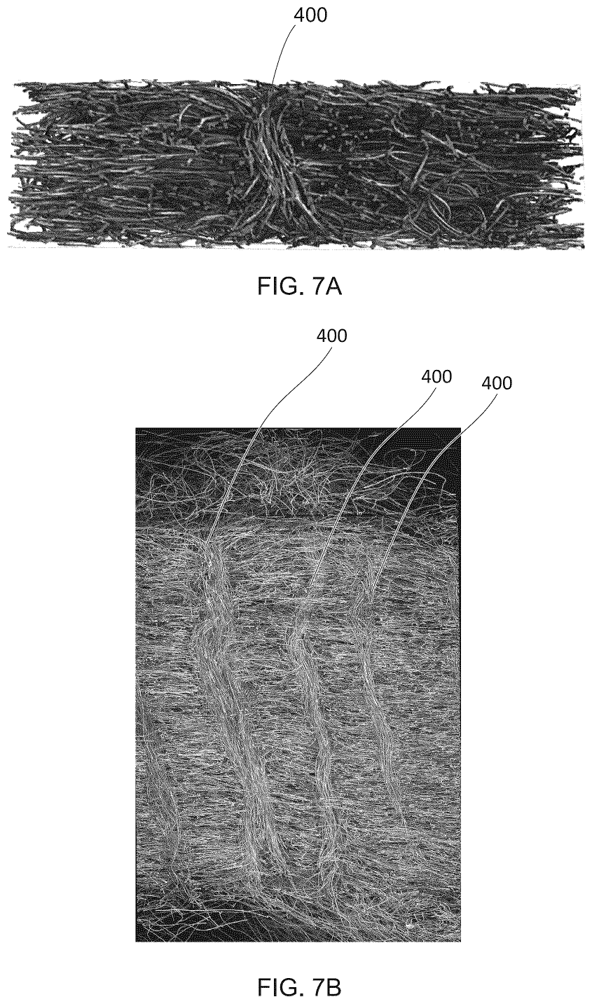

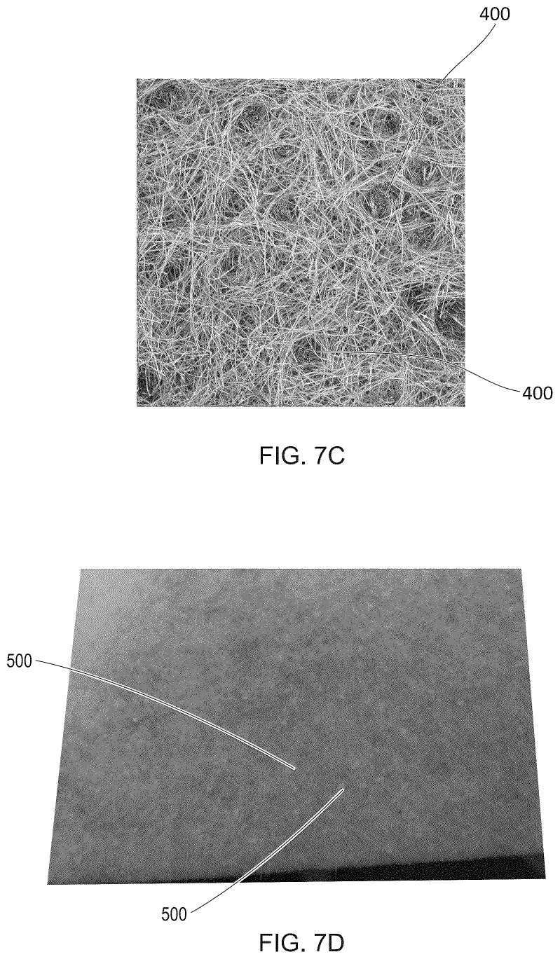

The precursor receiving web 247 comprising the layered carded web 235 of the first sublayer 120 and the layered carded web 245 of the second sublayer 130 such that either the layered carded web 235 is disposed on the layered carded web 245 or the layered carded web 245 is disposed on the layered carded web 235. The nonwoven precursor receiving web 247 is then needlepunched, with a needlepunching device 250, with the aid of one or more needle looms. The web of loose fibers, e.g., the layered precursor receiving web 247 of carded fibers, is converted into a structured non-woven web. The needlepunched fibers are mechanically oriented through the web. The needles can be arranged on a needle tool, e.g., a needle board or loom, in a non-lined arrangement. In the needle punching step at least one needle tool can be used in the range of from about 50 to 250, alternatively from about 70 to 200 and alternatively from about 90 to 180 needles per square inch. The precursor receiving web 247 is advanced to and through the need-punching device 250. A plurality of needles engage the precursor receiving web 247 to from a plurality of large-fiber integration areas. These large-fiber integration areas cause the fibers of the first sublayer and second sublayer to integrate forming the receiving layer web 255, which will later be cut to form the individual receiving layers 110 for the absorbent article. The needling reorients fibers from the x-y plane to the z-direction to create concentrated bundles of fibers oriented in the z-direction, such as illustrated in FIGS. 7A and 7B. The vertical bundles create pathways for fluid to flow efficiently through the material in the z-direction to reach the core faster, particularly in gush situations. The vertical fiber bundles 400 also increase resilience and compression resistance in the z-direction. FIG. 7C illustrates a top view of a portion of a receiving layer web 255 including the areas where the needles penetrate the web forming the vertical fiber bundles 400.

To further increase the ability of the fluid to flow through to the absorbent core, the receiving layer 110 can have small highly concentrated fiber areas spread throughout the layer. These small highly concentrated fiber areas, or capillary boosting points 500 can be seen in FIG. 7D. The capillarity boosting points have higher capillarity but as they are spread out over a wide area, they have minimal, or no, impact on overall permeability and hence minimal, or no, impact on the acquisition speed of the receiving layer 110. These capillary boosting points may be spaced apart in various ways. For instance, there may be from about 1 capillary boosting point to about capillary boosting points per square inch; alternatively, from about 3 to about 7 capillary boosting points per square inch. The capillary boosting points may be easily seen optically, especially with the use of a light table where the number of points is counted within a square inch of the material. The capillarity boosting points may vary in size from about 0.5 mm² to about 5 mm². The creation of the Capillary boosting points is controlled via careful calibration of the speeds within the carding unit and the speed of the needle punches combined with the small decitex fiber choices described herein.

The non-woven is then bonded via heat treatment after needling of the receiving layer web 225. This bonding of the bonding fibers creates a support matrix which enhances resiliency and stiffness of the receiving layer web 225. Absorbent articles that exhibit a soft cushiony feel, good resiliency, and fluid handling characteristics are contemplated herein. To have these characteristics, the caliper of the receiving layer web 225 is an important factor. Typical calipers of webs from conventional spunlace lines achieve a caliper factor (caliper per 10 gsm of basis weight) of 0.03 mm/gsm to 0.12 mm/gsm. In contrast, the receiving layer web 225 contemplated 20) herein can exhibit a caliper factor of at least 0.26 mm/gsm. The receiving layer web 225 contemplated herein has a caliper factor of between 0.26 mm/gsm to about 0.35 mm/gsm, including all values within these ranges and any ranges created thereby, according to the Caliper measurement method disclosed herein. The process as previously described achieves this relatively higher caliper.

Generally, it is desirable that the fibers forming the nonwoven receiving layer web be bonded following the carding/fiber laydown process and the crosslapping process, to impart a fabric-like structure and tensile strength (in both the MD and the CD) needed for the web to substantially retain its structure in downstream/later processes, and in the form of a receiving layer, during use by a user/wearer. The stiffening fibers are heat bonded such that the receiving layer 110 in the absorbent article has a MD/CD Peak Load ratio of from about 0.5 to about 1.75. Additionally, the degree of entanglement of the fibers by the needles also adds to the strength of the receiving layer. As an alternative to other methods of bonding such as mechanical compression spot bonding (with or without application of heating energy), adhesive bonding, etc., it has been found that bonding via air-through heating is effective for creating fiber-to-fiber bonds and imparting structure integrity to the web, while preserving inter-fiber pore/void size and loft, and imparting resiliency, to the nonwoven. In the heat bonding process, the heating temperature selection may be impacted, in part, by the constituent composition(s) of the stiffening fibers, the design and operating parameters of the heating equipment, and the web processing speed (i.e., duration of exposure to the heated environment). To impart uniform stiffness across the receiving layer web 225, the heating equipment and operating parameters should be set up to provide uniform heating to the receiving layer web 225. Even small variations in temperature can substantially impact the formation of fiber-to-fiber bonds between the bonding fibers and resulting tensile strength of the layer. An example of a suitable heat stiffening process that may be utilized is air-through heating, in which air is heated to the selected heating temperature and is blown and/or drawn (via vacuum) through the web along a direction that is approximately orthogonal to the larger planes defined by the web. In examples of suitable processes, air heated to the selected heating temperature is blown and/or drawn (via vacuum) through the carded fiber web as it is conveyed on a carrier belt along a machine direction, through an oven or heating chamber. When operating parameters including heating air temperature and velocity, and exposure time, are appropriately adjusted, a plurality of randomly distributed fiber-to-fiber bonds may be created within the fiber network, which impart structural integrity to the web. When constituent fibers are, for example, sheath-core bicomponent fibers in which the sheath component is a polymer having a melting temperature lower than that of the core component, the process may be configured such 20) that fusion bonds form between sheaths of adjacent contacting fibers without complete melting and loss of structure of the sheaths, while the cores remain in place, un-melted. In such process, the bonds may be formed without application of compression, and thus, without associated loss of caliper of the web and reduction in size of the inter-fiber pores/voids.

The receiving layer web 225 may then be wound in a rolled configuration and supplied to a converting manufacturing line where the receiving layer web 225 may be cut into individual receiving layers 110 and incorporated into an absorbent article.

Receiving layer 110 contemplated herein may be incorporated into a variety of absorbent articles. A non-limiting example of a schematic representation of an absorbent article in the form of a feminine hygiene pad as contemplated herein is illustrated and described herein with reference to FIGS. 3, 4A, and 4B. In summary, the pad 10 as contemplated herein may include a receiving layer 110, a backsheet 50, and an absorbent structure 40 disposed between the receiving layer 110 and the backsheet 50. The pad has a wearer-facing surface 64 and an opposing outward-facing or garment-facing surface 67. The wearer-facing surface 64 is formed primarily by the receiving layer 110 while the outward-facing surface 67 is formed primarily by the backsheet 50. Additional components (not shown) may be included proximate the wearer-facing surface 64 and/or the outward-facing surface 67. For example, if the absorbent article is an incontinence pad, a pair of barrier cuffs which extend generally parallel to a longitudinal axis of the pad 10 and may also form portions of the wearer-facing surface 64. Similarly, one or more deposits fastening adhesive (to be used by the user/wearer to affix the pad within her underwear, at an appropriate location, for use) may be present on the backsheet 50 and form a portion of the outward-facing surface 67 of the absorbent article.

### First Sublayer

As previously discussed, the receiving layer 110 comprises a first sublayer 120. The first sublayer 120 of the receiving layer 110 is liquid pervious and oriented towards and contacts the body of the wearer permitting bodily discharges to rapidly penetrate through it. The first sublayer, while being capable of allowing rapid transfer of fluid through it, may also provide for the transfer or migration of a lotion composition therefrom to facing surfaces of a wearer's skin. The first sublayer is formed of a nonwoven material as described herein. The first sublayer may include one strata of fibers or may be formed of more than one strata, which may comprise the same or different compositions. As described herein, the first sublayer is a carded nonwoven.

The first sublayer 120 as contemplated herein may be manufactured to have a basis weight of about 10 gsm to 60 gsm, more preferably about 15 gsm to 50 gsm, or most preferably about 20 gsm to 45 gsm, specifically reciting all values within these ranges and any ranges created thereby. For example, the first sublayer 120 may have a basis weight of from about 15 gsm to about 45 gsm. The range of desirable basis weight is influenced, at the lower end of the range, by the need for a level of web tensile strength required for processing, and by consumer preferences for a level of opacity and substantiality of loft, feel, and appearance. The range of desirable basis weight is influenced, at the upper end of the range, by the need for suitable rapid fluid acquisition and passage of fluid through the sublayer, and material cost concerns.

Nonlimiting examples of nonwoven materials suitable for use as the first sublayer 120 include fibrous materials made from natural fibers, e.g., cotton, including 100 percent organic cotton, modified natural fibers, semi-synthetic fibers (e.g., fibers spun from regenerated cellulose) synthetic fibers (e.g., fibers spun from polymer resin(s)), or combinations thereof. Synthetic fibers may include fibers spun from single polymers or blends of polymers.

However, in some examples it may be desired that nonwoven web of the first layer include less than 10 percent, more preferably less than 5 percent, and even more preferably less than 1 percent by weight of any combination of cotton fibers, other plant fibers, rayon fibers or monocomponent fibers comprising polyester or polyamide. Such fibers are often hydrophilic in nature and thereby may tend to cause the first layer to retain fluid rather than pass it along to absorbent components below creating a hygroscopic environment. For the same reason, such fibers may tend to cause the first sublayer to be prone to rewetting.

Synthetic fibers may include monocomponent fibers, bicomponent fibers or multicomponent fibers. (Herein, bi- or multicomponent fibers are fibers having cross sections divided into distinctly identifiable component sections, each formed of a single polymer or single homogeneous polymer blend, distinct from that of the other section(s). Such fibers and processes for making them are known in the art. Examples of bicomponent fiber configurations with substantially round cross sections include side-by-side or “pie slice” configurations, eccentric sheath-core configurations and concentric sheath-core configurations.

Nonwoven first sublayer contemplated herein may include fibers having myriad combinations of constituent chemistries. For example, fibers may be spun from thermoplastic polymeric materials, such as polyethylene (PE) and/or polyethylene terephthalate (PET). Fibers may be spun in the form of bi-component fibers. The bi-component fibers may have a core component of a first polymer (for example, PET) in combination with another polymer as a sheath component, in a sheath-core bicomponent configuration. In some embodiments, PE may form the sheath component in combination with a PET core component. Fibers that include a PET component may be selected to help provide bulk and resilience and a resulting cushiony feel to the nonwoven web. Additionally, fibers that include a PET component, having resilience, help the web retain the area and dimensions of apertures created therethrough, if included.

Other polymeric materials may be included. For example, fibers spun of polypropylene, polyethylene, co-polyethylene terephthalate, co-polypropylene, and other thermoplastic resins may be included. It may be desired that the polymer with the lower melting temperature form the sheath component where sheath-core bi-component fibers are included. Additionally, without wishing to be bound by theory, it is believed that the use of polyethylene terephthalate as a core component can help impart resilience to the fiber, and as a result, to the receiving layer 110.

Polyethylene, as a polymer component from which fibers may be spun, has a relatively lower melting temperature, and exhibits a relatively slick/silky surface feel as compared with other potentially useful thermoplastic polymers. PET has a relatively higher melting temperature and exhibits relatively greater stiffness and resiliency. Accordingly, in some examples the sublayer nonwoven fibers that are of a sheath-core bicomponent configuration may be desired, in which the sheath component is predominantly polyethylene, and the core component is predominantly PET. The polyethylene is useful for imparting the fibers and thus the receiving layer with a silky feel, and for enabling inter-fiber bonding via heat treatment that causes sheaths of adjacent/contacting fibers to melt and fuse at the lower melting temperature of the polyethylene, while the PET is useful for imparting resilience, and will not melt at lower temperatures that will melt PE in the heat treatment process. The inventors have found that a suitable weight ratio in such PE/PET sheath-core bicomponent fibers may be about 40:60 to about 60:40.

Depending upon the chemical composition thereof, surfaces of fibers will be, inherently, either hydrophilic or hydrophobic, to varying extents. For example, surfaces of fibers spun or otherwise formed from some types of polymers such as polyethylene and polypropylene will be, inherently, hydrophobic. In contrast, surfaces of other types of fibers, such as rayon fibers, will be inherently hydrophilic. Surfaces of natural fibers may be inherently hydrophilic or hydrophobic, but this may depend upon the processing the fibers have undergone. For example, cotton fibers as harvested bear coatings of natural oils and/or waxes and as such their surfaces are hydrophobic. After they have undergone processes including scouring and bleaching, however, the oils and/or waxes will have been stripped away, rendering the fiber surfaces hydrophilic.

Manufacturers and/or suppliers of spun synthetic staple fibers currently apply coatings, in the form of surface finishing agents or processing aids, to the fibers, for purposes of providing lubricity in, for examples, the carding processes. These surface finishing agents or processing aids may be formulated to be either hydrophobic or hydrophilic, and to be substantially durable for purposes herein, in that they will not dissolve in aqueous fluids over the ordinary duration of wear of an absorbent article. Thus, a manufacturer or supplier of spun synthetic staple fibers may offer fibers with either hydrophobic or hydrophilic surface finishes.

Noting that spun synthetic staple fibers may be obtained with either inherently hydrophobic or hydrophilic surfaces, or obtained with surface finishes that render their surfaces hydrophilic or hydrophobic, it may be desirable to choose fibers with surfaces that are either hydrophilic (“hydrophilic fibers”) or hydrophobic (“hydrophobic fibers”), or choose a blend of fibers of both types. In some examples it may be preferable that the fiber constituents of the first sublayer be, by weight, predominantly, substantially, or entirely hydrophobic, or rendered hydrophobic via fiber surface finish. A first sublayer formed of a nonwoven web with predominately hydrophobic fiber constituents will be resistive to rewetting. Alternatively, if the sizes of the pores or inter-fiber voids within the fibrous structure of such nonwoven web are not sufficiently large, the first sublayer may resist the passage of fluid from the wearing facing surface through to the absorbent core components of the article there beneath, i.e., will not readily/rapidly acquire fluid, unless other features are included in combination, as described herein.

The fibers constituting portions, a majority (by surface area), or all, of the section of web material from which of the first sublayer is formed, may be a blend of both hydrophobic fibers and hydrophilic fibers. In such examples, the hydrophilic fibers can serve to help wick fluid from the wearer-facing surface of the first sublayer down to the second sublayer and the absorbent core components beneath, while the hydrophobic fibers can serve to help the first sublayer resist rewetting. The inventors have discovered that a successful balance may be advantageous for performance of the pad, wicking fluid away from the skin and avoiding rewet. Accordingly, in some examples the first sublayer nonwoven may include a mix of hydrophobic and hydrophilic fibers. For example, the nonwoven may include at least about 40 percent, more preferably at least about 50 percent, or most preferably at least about 60 percent hydrophilic fibers by weight of the fibers, specifically including all values within these ranges and any ranges created thereby. In more particular examples, the nonwoven first sublayer may comprise about 40 percent to 70 percent, more preferably about 45 percent to 68 percent, or most preferably from about 50 percent to 65 percent, by weight, hydrophilic fibers, specifically reciting all values within these ranges and any ranges created thereby. The first sublayer nonwoven may include a blend of hydrophilic fibers and hydrophobic fibers in a weight ratio of hydrophilic fibers to hydrophobic fibers of 30:70 to 70:30, more preferably 35:65 to 65:35, and even more preferably 40:60 to 60:40. As noted above, the hydrophilicity of the hydrophilic fibers may be affected by application of a surface treatment composition. Without wishing to be bound by theory, it is believed that where a majority of the fibers are hydrophilic, fluid acquisition speed can be improved by combination with other features described herein, while not overly impacting rewet in a negative or unacceptably negative manner. Where less rewet is the goal, then the converse may be true. In this circumstance, a higher weight fraction of hydrophobic fibers may be desired.

Fibers are typically manufactured, selected and purchased by linear density specification, such expressed as denier or decitex. For fibers of a given polymer constitution, linear density correlates with fiber size/diameter. The fibers constituting the first sublayer may be selected to have an average linear density of about 1.0 to 3.0 denier, more preferably about 1.5 to 2.5 denier, and even more preferably about 1.8 to 2.2 denier, and all combinations of subranges within these ranges are contemplated herein. Fibers with varying linear densities within the ranges set forth above may be selected and included as well.

In other examples, the fibers constituting the first layer may be selected to have an average linear density of about 3.0 to 5.0 denier, more preferably about 3.5 to 4.5 denier, and even more preferably about 3.8 to 4.2 denier, and all combinations of subranges within these ranges are contemplated herein. It has been learned that fibers selected within these ranges, in combination with other features disclosed herein, may be deemed to constitute a first sublayer material of acceptable softness to many consumers, as well as to provide other advantages over smaller fibers.

One advantage is that the relatively larger fibers generally provide a nonwoven web material with relatively larger inter-fiber/intra-web spaces or voids therewithin, thereby providing larger passageways through which fluid may more rapidly travel through the nonwoven from the wearer-facing side through to the outward-facing side (and thus to absorbent components below). Additionally, although relatively larger fibers of a given composition are stiffer than smaller fibers of similar composition, which may somewhat compromise surface “softness” attributes, the greater fiber stiffness can also enhance a feeling of greater resiliency, springy or cushiony feel to the first layer nonwoven.

The first sublayer 120 may comprise staple fibers having a length of at least about 30 mm, 40 mm, or 50 mm, up to about 75 mm, or about 30 to 75 mm, or about 35 to 55 mm, reciting for said range every 1 mm increment therein. In some embodiments, the staple fibers may have a length of about 38 mm. The first sublayer may have a mean pore size of from about 80 μm to about 150 μm, as determined by the Micro-CT Pore Size Measurement Method.

### Second Sublayer

The second sublayer 130 of the receiving layer 110, which is positioned between the first sublayer 120 and the absorbent core 45. The second sublayer 130 adds caliper to the absorbent article and is typically compressible, and resilient, which can impart a feeling of softness and/or a “cushiony” feel to the article, which is due, at least in part, to the resiliency of the sublayers. The absorbent articles contemplated herein exhibit good resiliency properties both in dry and wet conditions. The second sublayer described herein is a nonwoven comprising cellulosic and bonding 20) fibers and has a caliper of from about 0.26 mm to about 0.35 mm at 2 kPa. The nonwoven fibers of the second sublayer 130 have a decitex below about 2. More specifically, at least one of the cellulosic fibers, the bonding fibers, and the divider fibers have a decitex less than about 2. To maintain this relatively higher caliper of the second sublayer and the receiving layer, the second sublayer must include finer fibers (decitex less than about 2) to maintain the desired pore size so that fluid is quickly transferred to the absorbent core and is trapped within the core to prevent rewet. Because of these finer fibers (decitex less than about 2) and the need to maintain caliper or thickness of the layer, needlepunching as previously described is an ideal process because it does not as aggressively adversely affect the caliper. The needles are able to move and integrate the finer fibers without compressing the layer, which allows the caliper to be maintained. These finer fibers also allow for the micro-integration due to their smaller size and ability to easily move into open spaces to create that integration among the sublayers and prevent gaps being formed between the sublayers.

The second sublayer 130 may include a nonwoven having a basis weight of from about 40 gsm to about 60 gsm. The second sublayer 130 may have a basis weight of up to 75 grams per square meter (gsm); or a basis weight of up to 70 gsm; or a basis weight in the range of about 40 gsm to about 75 gsm; or in the range of about 50 gsm to about 70 gsm; or in the range of about 55 gsm to about 65 gsm, including any values within these ranges and any ranges created thereby.

The second sublayer 130 may be any suitable shape including but not limited to oval, a stadium, rectangle, an asymmetric shape, peanut, trapezoid, rounded trapezoid, ovoid, nested and hourglass. In some examples, the second sublayer may have a contoured shape, e.g., one that is narrower in the longitudinally intermediate region than in the end regions. In other examples, the second sublayer may have a tapered shape that is a wider in one end region of the pad, and tapers to a narrower width in the other end region of the pad. The second sublayer may have a long oval shape. The second sublayer may have a nested shape where one end is concave, and the other end is convex. The second sublayer may be the same shape as the first sublayer.

In addition to the softness and resiliency benefits due to the composition of the fibers and structural attributes of the sublayers contemplated herein, stain size control and faster fluid acquisition may be obtained. Stain size is important in the way the absorbent article is perceived by the user. For feminine hygiene pads, when a stain visible on the pad after a duration of use/wear is relatively large along x-y directions, users may perceive that the pad is near failure based on the appearance of the stain and its proximity to the outer periphery of the pad. In contrast, a smaller, lighter stain can have a reassuring effect on the user/wearer, by creating a perception that the pad is not near failure because the edges of the stain lie substantially longitudinally and/or laterally short of the outer periphery of the pad. The ability of the sublayers to pull fluid toward the garment-facing surface or away from the wearer-facing surface results in relatively reduced stain size. The structure of the sublayers allows for fluid to be more readily pulled away from the wearer-facing surface.

Additionally, fluid acquisition speed of the pad may be deemed important to the user/wearer, as rapid acquisition can help make the user/wearer feel dry and clean. When the pad requires a relatively long time to drain discharged fluid from the first sublayer, it can cause the user to feel wetness, and feel unclean. To enable rapid acquisition/intake of discharged fluid, but also sufficient capillarity to dewater the first sublayer, the second sublayer has a particular pore volume range. The second sublayer can have an average pore size of from about 90 to about 330 μm, alternatively the mean pore size can be from about 100 to about 150 μm, as determined by the Micro-CT Pore Size Measurement Method. The second sublayer mean pore size is less than the first sublayer mean pore size to control the acquisition and transfer of fluid to the absorbent core.

The second sublayer can draw fluid through and from the first sublayer via capillary action or wicking forces, of sufficient magnitude to overcome any resistance to passage of the fluid through the first sublayer, or attraction the first sublayer may have for the fluid, that may be present as a result of the composition and/or configuration of the first sublayer. The receiving layer also can accept and contain a gush of fluid by providing pore volume as a temporary reservoir, together with distribution functions, to efficiently utilize the absorbent structure, give it time to imbibe and absorb the fluid.

The inventors have found that to deliver the desired levels of softness and resiliency combined with the need for rapid acquisition and high capillarity needs, that the second sublayer breaks technology limitations of the past. The second sublayer of the present invention delivers high caliper, that is resilient and yet flexible combined with high capillarity and permeability.

Overall, the second sublayers of the present disclosure may comprise from about 15 percent to about 35 percent by weight, from about 20 percent to about 35 percent by weight, from about 25 percent to about 35 percent by weight of the second layer of cellulose, specifically including any values within these ranges and any ranges created thereby of cellulosic fibers. In one specific example, second sublayers may comprise about 30 percent by weight of cellulosic fibers. Suitable cellulosic fibers include cotton, rayon, viscose, lyocell, natural cellulose, regenerated cellulose and combinations thereof. Particularly suitable cellulosic fibers include viscose. The cellulosic fibers have a decitex of below about 2; alternatively, from about 0.5 to about 1.7. In some embodiments, the second sublayer will have a greater percentage by weight of cellulosic fibers than the first sublayer.

The cellulosic fibers of the second sublayer may have any suitable cross-section profile shape (where the cross-section lies along a plane that is perpendicular with the greater length dimension of the fiber when it is straight). Some examples of suitable shapes may include trilobal, “H,” “Y,” “X,” “T,” round, or flat ribbon. Further, the absorbing fibers can have cross sections that are solid, hollow, or combinations of hollow and solid. Other examples of suitable multi-lobed, cellulosic fibers for utilization in the second sublayer described herein are disclosed in U.S. Pat. Nos. 6,333,108; 5,634,914; and 5,458,835. A trilobal fiber shape can improve wicking and improve opacity and stain concealment properties. Suitable trilobal rayon fibers are available from Kelheim Fibres GmbH (Kelheim, Germany) and sold under the trade name GALAXY. While each layer may include a different shape of absorbing fiber, much like mentioned above, not all carding equipment may be suited to handle such variation between/among layers. In some embodiments, the second sublayer may include cellulosic fibers having a round (circular) shape.

The cellulosic fibers may include any suitable absorbent material. Suitable absorbent fibrous materials include cotton, cellulose (e.g., wood) pulp, regenerated cellulose (rayon, viscose, lyocell, etc.) or combinations thereof. In one example, the second sublayer may include viscose fibers.

The staple length of the cellulosic fibers may be selected to be about 20 mm to 100 mm, or 30 mm to 50 mm, or even 35 mm to 45 mm, specifically reciting all values within these ranges and any ranges created thereby. In general, the fiber length of wood pulp is from about 4 to 6 mm and cannot used in conventional carding machines because the pulp fibers are too short. Accordingly, if wood pulp is desired as a fiber in the second layer, additional processes to blend and add pulp to the carded webs may be beneficial. In some examples, pulp may be airlaid between carded webs with the combination being subsequently integrated. As another example, tissue made from pulp may be utilized in combination with the carded webs and the combination may be subsequently integrated.

Similarly, overall, the second sublayer may comprise from about 65 percent to about 85 percent by weight of the second sublayer of bonding fibers, specifically reciting all values within these ranges and any ranges created thereby. Suitable bonding fibers include bicomponent polyethylene terephthalate/polyethylene, combinations of polyethylene, polypropylene, polyethylene terephthalate, Co-polyethylene terephthalate and combinations thereof. The bonding fiber can be polyethylene terephthalate/polyethylene wherein the core is polyethylene 20) terephthalate, and the sheath is polyethylene. The bonding fibers may comprise bicomponent fibers. Particularly suitable bonding fibers can comprise polymeric fibers. The bonding fibers can have a decitex of less than about 2, alternatively from about 1 to about 2. The bonding fibers enhance the ability of the second layer to recover its shape and/or caliper following application of compressive loads that are imposed during use. Stated another way, the bonding fibers provide resiliency to the receiving layer.

The concentration of bonding fibers in the first sublayer and the second sublayer are related. If the amount of bonding fibers in the first sublayer is significantly different than the second sublayer, this can result in one sublayer being substantially stiffer than the other sublayer and for imparting unwanted stiffness into the sublayer. The sublayers are integral layers so that when processing, these sublayers are processed together. More specifically, when the sublayers are heated to bond the bonding fibers, the sublayers are heated within the same oven for the same amount of time. However, given the different fluid handling properties needed in the first sublayer in comparison to the second sublayer, the bonding fibers need to be selected to maintain an open structure resulting in an optimized capillary change in the first sublayer and to maintain a larger capillary structure in the second sublayer to move fluid away from the topsheet. The denier and amount of the bonding fibers may be selected to optimize the fluid handling and stiffness properties of the layer. For example, in some embodiments, the first sublayer may including from about 80 to 100 percent by weight bonding fibers that are bicomponent fibers having about 4.0 denier, and the second sublayer may include from about 10 to 30 percent by weight of bonding fibers that are bicomponent fibers having about 2.0 denier. In some embodiments, the first sublayer will have a greater percentage by weight of bonding fibers having a larger denier fiber than the second sublayer. Stated another way, the second sublayer will have a greater percentage by weight of smaller denier fibers than the first sublayer.

The second sublayer may also comprise from about 35 percent to about 55 weight percent of the second sublayer of divider fibers. Suitable divider fibers include polypropylene, polyethylene terephthalate, bicomponent polyethylene, bicomponent polypropylene, bicomponent polyethylene terephthalate and combinations thereof. Suitable divider fibers have a decitex of less than about 2, alternatively from about 0.5 to about 2. Particularly suitable divider fibers may comprise non-cylindrical polymeric fibers including but not limited to polypropylene. The divider fibers function to divide spaces in between the bonding and cellulosic fibers thereby creating smaller pore sizes that drive capillarity. The small size and optional non-cylindrical shape further enhance the capillarity. In some embodiments, the second sublayer will have a greater percentage by weight of divider fibers than the first sublayer.

The cellulose, bonding, and/or divider fibers can have a length of from about 10 mm to about 120 mm, alternatively from about 24 mm to about 95 mm, and alternatively from about 36 mm to about 75 mm. The cellulose, bonding, and divider fibers each have a fiber length and the fiber lengths of each of these types of fibers may be the same length, a different length, or a combination thereof. The weight fractions of cellulosic fibers, bonding fibers, and/or divider fibers may be determined via the Material Compositional Analysis method disclosed below.

In order to deliver the needed nonwoven caliper and the necessary pore structure for fluid performance it is necessary to utilize low decitex fibers as previously disclosed. It is additionally important to utilize a process that imparts material strength in both the MD and CD directions. This is particularly difficult as improper integration methods of the fibers will collapse the overall material caliper, especially since the fiber decitex are so low. The second layer comprises a large-fiber integrated comprises a stitch density of between 90 and 220 punches per square centimeter. The stitch direction is selected from the top, bottom, and combinations thereof. Additionally, the second layer has an MD:CD fiber orientation from about 1:1 to about 1:1.75.

The second sublayer may have a MD peak load of from about 4 to about 85 Newtons and may also have a CD peak load of from about 4 to about 130 Newtons. The second sublayer 130 can have a caliper factor of between 0.26 mm to about 0.35 mm, including all values within these ranges and any ranges created thereby. The caliper and caliper factor of the second sublayer of the present disclosure may be determined by the Caliper and Caliper Factor test methods disclosed herein.

Further, in order to enhance the stabilizing effect of the integration, crimped fibers may be utilized. As discussed in additional detail below, the second sublayer and, thus, the receiving layer of the present disclosure may comprise cellulose fibers, bonding fibers, and additionally may comprise divider fibers. One or more of these fibers may be crimped prior to integration. For example, where synthetic fibers are utilized, these fibers may be mechanically crimped via intermeshing teeth. And for the cellulosic fibers, these fibers may be mechanically crimped and/or may have a chemically induced crimp due to the variable skin thickness formed during creation of the cellulosic fibers.

### Absorbent Structure

The absorbent structure 40 of the present disclosure may have any suitable shape including but not limited to oval, a stadium, rectangle, an asymmetric shape, peanut, trapezoid, rounded trapezoid, ovoid, nested and hourglass. In some examples, absorbent structure 40 may have a contoured shape, e.g., one that is narrower in the longitudinally intermediate region than in the end regions. In other examples, the absorbent structure may have a tapered shape that is a wider in one end region of the pad, and tapers to a narrower width in the other end region of the pad. The absorbent structure may have a nested shape where one end is concave, and the other end is convex. The absorbent structure 40 may have varying stiffnesses in the MD and CD.

The configuration and construction of the absorbent structure 40 may vary (e.g., the absorbent structure 40 may have varying caliper zones, a hydrophilic gradient, a superabsorbent gradient, or lower average density and lower average basis weight acquisition zones). Further, the size and absorbent capacity of the absorbent structure 40 may also be varied to accommodate a variety of wearers. However, the total absorbent capacity of the absorbent structure 40 should be compatible with the design loading and the intended use of the disposable absorbent article or incontinence pad 10.

In some examples, the absorbent structure 40 may include a plurality of layers each having particular features and/or functions. Are some examples, the absorbent structure 40 may include a wrap (not shown) included to envelope enveloping the absorbent constituents of the absorbent structure. The wrap may be formed by one or more nonwoven materials, tissues, films or other materials, or laminates thereof. In one form, the wrap may be formed of only a single material, substrate, laminate, or other material that is wrapped at least partially around itself.

The absorbent structure 40 may include one or more adhesives, for example, to help immobilize the SAP or other absorbent materials within the first and second laminates. Suitable absorbent structures comprising relatively high amounts of superabsorbent polymer (“SAP”—also known as “absorbent gelling material,” or “AGM”) with various core designs are disclosed in U.S. Pat. No. 5,599,335; EP 1 447 066; WO 95/11652; US 2008/0312622 A1; and WO 2012/052172

Additions to the absorbent structure are contemplated. Potential additions to the absorbent structure are described in U.S. Pat. Nos. 4,610,678; 4,673,402; 4,888,231; and 4,834,735. The absorbent structure may further include layers that mimic the dual core system containing an acquisition/distribution core of chemically stiffened fibers positioned over an absorbent storage core as described in U.S. Pat. No. 5,234,423; and in U.S. Pat. No. 5,147,345. These may be deemed useful to the extent they do not negate or conflict with the effects of the below described laminates of the absorbent structure of the present invention.

Some further examples of a suitable absorbent structures 40 that can be used in the absorbent article of the present disclosure are described in US 2018/0098893 and US 2018/0098891. The absorbent structure 40 may include conventional absorbent materials. In addition to conventional absorbent materials such as creped cellulose wadding, fluffed cellulose fibers, rayon or viscose fibers and comminuted wood pulp fibers (also known as airfelt or fluff pulp), and textile fibers, the storage layer may also include particles or fibers of superabsorbent material that imbibes fluids and forms hydrogels. (Such materials are also known as absorbent gelling materials (AGM).) AGM is typically capable of absorbing a relatively large weight quantity of body fluid per dry weight AGM, retaining it under moderate pressure. Synthetic fibers spun from polymers such as cellulose acetate, polyvinyl fluoride, polyvinylidene chloride, acrylics (such as ORLON), polyvinyl acetate, non-soluble polyvinyl alcohol, polyethylene, polypropylene, polyamides (such as nylon), polyesters, bi-component fibers, tricomponent fibers, mixtures thereof and the like can also be included in the secondary storage layer. The absorbent structure 40 may also include filler materials, such as PERLITE, diatomaceous earth, VERMICULITE, or other suitable materials, that can serve to reduce changes of rewetting.

The absorbent structure 40 may include absorbent gelling material (AGM) in a uniform distribution throughout or may include it in a non-uniform distribution. The AGM may be distributed and/or concentrated via deposit thereof into channels or pockets, or may be deposited in patterns including stripes, crisscross patterns, swirls, dots, or any other pattern, either two or three dimensional, that can be imagined. The AGM may be sandwiched between a pair of fibrous cover layers. AGM may be encapsulated, at least in part, by a single fibrous cover layer.

Portions of the absorbent structure 40 may be formed substantially only of superabsorbent material/AGM, or may be formed of AGM distributed and dispersed in a suitable carrier structure such as a batt or accumulation of cellulose fibers in the form of fluff or stiffened fibers. One non-limiting example of a storage layer may include a first layer formed substantially only of AGM particles or fibers, that are placed or deposited onto a second layer that is formed of a distribution of AGM particles or fibers, within cellulose fibers.

Examples of absorbent structures formed of layers of superabsorbent material/AGM and/or layers of superabsorbent material/AGM dispersed within a batt or other accumulation of cellulose fibers, that may be utilized in the absorbent articles (e.g., sanitary napkins, incontinence products) contemplated herein are disclosed in US 2010/0228209A1. Absorbent structures comprising relatively high amounts of SAP/AGM with various core designs are disclosed in U.S. Pat. No. 5,599,335; EP 1 447 066; WO 95/11652; US. 2008/0312622 A1; WO 2012/052172; U.S. Pat. Nos. 8,466,336; and 9,693,910 to Carlucci. These may be used to configure the absorbent structure, also referred to herein as an absorbent core or absorbent core layer.

### Backsheet

The backsheet 50 may be disposed beneath the absorbent structure 40 and be the outwardmost layer of the article, forming the outward-facing or garment-facing surface of the article. The backsheet 50 may be joined to the absorbent structure 40 and/or to the receiving layer 110 (about the outer periphery or a portion thereof) by any suitable attachment methods known in the art. For example, the backsheet 50 may be secured to the absorbent structure 40 by a uniform continuous layer of adhesive, a patterned layer of adhesive, or an array of separate lines, spirals, or spots of adhesive. Alternatively, the attachment methods may comprise using heat bonds, pressure bonds, ultrasonic bonds, dynamic mechanical bonds, or any other suitable attachment methods or combinations of these attachment methods as are known in the art.

The backsheet may be impervious, or substantially impervious, to liquids (e.g., urine, menstrual fluid) under ordinary conditions of use, and may be manufactured from a thin plastic film, although other flexible liquid impervious materials may also be used. The backsheet may prevent, or at least inhibit, exudates absorbed and contained in the absorbent structure from wetting underwear, outer clothing, bedding, etc. which may come into contact with or proximity to the article. However, in some examples the backsheet may be configured so as permit vapor to escape from the absorbent structure (i.e., is “breathable”) while in examples the backsheet may be configured so as to be vapor-impermeable (i.e., non-breathable). Backsheet may include a polymeric film such as a film of polyethylene or polypropylene. A suitable material for the backsheet is a thermoplastic film having a thickness of approximately 0.012 mm (0.5 mil) to 0.051 mm (2.0 mils), for example. Suitable materials for the backsheet film may have a basis weight of from about 8 to about 25 gsm. Any suitable liquid impermeable backsheet known in the art may be utilized with the present invention.

The backsheet serves as a barrier to prevent migration of fluids absorbed and retained in the absorbent structure, to the outward-facing surface of the pad. Suitable materials are soft, smooth, compliant, and vapor pervious material that provides for softness and conformability for comfort and is low noise producing so that movement does not cause unwanted sound. Non-limiting examples of materials suitable for forming backsheets are described in U.S. Pat. Nos. 5,885,265; 6,462,251; 6,623,464; and 6,664,439. Examples of suitable dual- or multi-layer breathable backsheets include those described in U.S. Pat. Nos. 3,881,489; 4,341,216; 4,713,068; 4,818,600; EP 203 821; EP 710 471; EP 710 472; and EP 793 952. Additional examples of suitable single layer breathable backsheets include those described in GB A 2184 389; GB A 2184 390; GB A 2184 391; U.S. Pat. Nos. 4,591,523; 3,989,867; 3,156,242; and WO 97/24097.

The backsheet may be a nonwoven web having a basis weight of about 20 gsm to 50 gsm. In one example, the backsheet may be a hydrophobic 23 gsm spunbond nonwoven web of 4 denier polypropylene fibers, available from Fiberweb Neuberger, under the trade designation F102301001. The backsheet may be coated with a non-soluble, liquid swellable material as described in U.S. Pat. No. 6,436,508.

The backsheet has an outward-facing side and an opposing wearer-facing side. The outward-facing side of the backsheet may include a non-adhesive area and an adhesive area. The adhesive area may be provided by any conventional means, for the purpose of enabling the user/wearer to affix the pad to the wearer-facing surface of her underwear at a location suitable for use. Pressure-sensitive adhesives have been found to work well for this purpose.

### EXAMPLES

As previously discussed, the inventive receiving layer 110 is a carded, crosslapped, and needlepunched nonwoven. By manufacturing the receiving layer 110 as described herein, by laying the first sublayer over the second sublayer in the manufacture of the web enables very close association of the fibers not possible via typical gluing of materials together. The gaps between the first sublayer and the second sublayer are minimized and/or eliminated resulting in relatively improved performance of the absorbent article. The difference in fiber sizes and types between the first sublayer and the second sublayer better enables the fibers to intermingle during the manufacturing process. It is to be appreciated that the difference between the fiber denier of the first sublayer and the fiber denier of the second sublayer is at least 15% or at least 25% or from about 15% to about 100% or from about 24% to about 95%. The first mean pore size is greater than the second mean pore size. The second mean pore size may be from about 40% to about 90% or from about 50% to about 85% or from about 60% to about 80% of the first mean pore size.

Inventive Sample 1: The receiving layer having a total basis weight of 80 gsm. The first sublayer is a nonwoven having a basis weight of 20 gsm and a caliper of 0.55 mm as measured at 0.5 kPa prior to winding. The first sublayer having 40 percent by weight polyethylene terephthalate/polyethylene bicomponent fibers that are hydrophobic and 60 percent by weight polyethylene terephthalate/polyethylene bicomponent fibers that are hydrophilic. The fibers of each of the first sublayer have a decitex (dtex) of 4.4. The second sublayer is a nonwoven having a basis weight of 60 gsm and a caliper of 1.65 mm as measured at 0.5 kPa prior to winding. The second sublayer having 25 percent by weight viscose cellulose fibers having a 0.9 dtex; 45 percent by weight tri-lobal polypropylene fibers having a 1.0 dtex; and 30 percent by weight bi-component fibers having 1.7 dtex. The bicomponent fibers including polyethylene terephthalate and polyethylene in a core-sheath configuration where the polyethylene is the sheath and the polyethylene terephthalate is the core.

Inventive Sample 2: The receiving layer having a total basis weight of 80 gsm. The first sublayer is a nonwoven having a basis weight of 40 gsm. The first sublayer having 25 percent by weight viscose cellulose fibers having a 1.3 dtex and 75 percent by weight polyethylene terephthalate/polyethylene bicomponent fibers having a 1.7 dtex. The second sublayer is a nonwoven having a basis weight of 60 gsm and having 25 percent by weight viscose cellulose fibers having a 0.9 dtex; 45 percent by weight tri-lobal polypropylene fibers having a 1.0 dtex; and 30 percent by weight bi-component fibers having 1.7 detex. The bicomponent fibers including polyethylene terephthalate and polyethylene in a core-sheath configuration where the polyethylene is the sheath and the polyethylene terephthalate is the core.

For Inventive Sample 1 and Inventive Sample 2, the receiving layer was constructed using the same carding and needlepunching process described above and assembled into inventive products 1 and 2 respectively. Inventive Samples 1 and 2 include the same backsheet. The backsheet is 12 gsm polypropylene film, available from RKW. Inventive Samples 1 and 2 contain the same absorbent core. The absorbent core is an airlaid absorbent core comprising pulp fibers, absorbent gelling material, and bico fibers, having a basis weight of 150 gsm available from Glatfelter, York, Pa., USA. The inventive receiving layers, Samples 1 and 2, are combined with the backsheet and absorbent core to form inventive products.

As previously discussed, the first sublayer and the second sublayer may have material properties that allow for superior fluid handling while providing a cushy and comfortable absorbent article to the consumer. The following data table provides data on the caliper, average pore diameter, and through plane permeability of an inventive sample of the first sublayer and the second sublayer as the raw material, meaning the web material prior to incorporation into the product, and the first sublayer and second sublayer within the finished product. This data was generated under a pressure of 2 kPa.

| | | Raw Material | Finished | Finished |
|---|---|---|---|---|
| | Raw Material | Second | Product Second | Product First |
| | First Sublayer | Sublayer | Sublayer | Sublayer |
| Caliper [mm] | 0.24 | 0.892 | 0.78 | 0.25 |
| Mean Pore Size [μm] | 123 | 73 | 68 | 97 |
| Through Plane | 354.6 | 152.0 | 111.5 | 202.6 |
| Permeability [Darcy] | | | | |

### Test Methods

Fiber Decitex (dtex)

Textile webs (e.g., woven, nonwoven, airlaid) are comprised of individual fibers of material. Fibers are measured in terms of linear mass density reported in units of decitex. The decitex value is the mass in grams of a fiber present in 10,000 meters of that fiber. The decitex value of the fibers within a web of material is often reported by manufacturers as part of a specification. If the decitex value of the fiber is not known, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning calorimetry), and then using a literature value for density of the composition to calculate the mass in grams of the fiber present in 10,000 meters of the fiber. All testing is performed in a room maintained at a temperature of 23° C.±2.0° C. and a relative humidity of 50%±2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is about 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bicomponent, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area aₖ for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g., FTIR spectroscopy and DSC) may be required to fully characterize the fiber composition. Repeat this process for each fiber type present in the web material.

The decitex dₖ value for each fiber type in the web material is calculated as follows:

$${d}_{k} = 10000 m \times {a}_{k} \times {ρ}_{k} \times {10}^{\_ 6}$$

where dₖ is in units of grams (per calculated 10,000 meter length), aₖ is in units of μm², and ρₖ is in units of grams per cubic centimeter (g/cm³). Decitex is reported to the nearest 0.1 g (per calculated 10,000 meter length) along with the fiber type (e.g., PP, PET, cellulose, PP/PET bico).

The denier value for each fiber type in the web material is calculated as follows:

$$denier = 0.9 \times {d}_{k}$$

where dₖ is in units of grams (per calculated 10,000 meter length) and denier is in units of grams. Denier is reported to the nearest 0.1 g along with the fiber type (e.g., PP, PET, cellulose, PP/PET bico).

### Basis Weight

The basis weight of a test sample is the mass (in grams) per unit area (in square meters) of a single layer of material and is measured in accordance with compendial method WSP 130.1. The mass of the test sample is cut to a known area, and the mass of the sample is determined using an analytical balance accurate to 0.0001 grams. All measurements are performed in a laboratory maintained at 23° C.±2° C. and 50%±2% relative humidity and test samples are conditioned in this environment for at least 2 hours prior to testing.

Measurements are made on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen is as large as possible so that any inherent material variability is accounted for.

Measure the dimensions of the single layer test specimen using a calibrated steel metal ruler traceable to NIST, or equivalent. Calculate the Area of the test specimen and record to the nearest 0.0001 square meter. Use an analytical balance to obtain the Mass of the test specimen and record to the nearest 0.0001 gram. Calculate Basis Weight by dividing Mass (in grams) by Area (in square meters) and record to the nearest 0.01 grams per square meter (gsm). In like fashion, repeat for a total of ten replicate test specimens. Calculate the arithmetic mean for Basis Weight and report to the nearest 0.01 grams/square meter.

### Caliper

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2° C. and 50%±2% relative humidity and test specimens are conditioned in this environment for at least 2 hours prior to testing.

Caliper is measured with a manually-operated micrometer equipped with a pressure foot capable of exerting a steady pressure of 0.50 kPa±0.01 kPa onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it is larger than the pressure foot.

To measure caliper, first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the caliper of the test specimen to the nearest 0.001 mm. In like fashion, repeat for a total of ten replicate test specimens. Calculate the arithmetic mean for all caliper measurements and report as Caliper to the nearest 0.001 mm.

### Caliper Factor

The caliper factor, as mentioned previously is the caliper per 10 gsm of basis weight of the sample. So, the equation is caliper/(basis weight/10).

### Density

Density is calculated based upon the basis weight and caliper with appropriate unit conversion to arrive at g/cc.

### Material Compositional Analysis

The quantitative chemical composition of a test specimen comprising a mixture of fiber types is determined using ISO 1833-1. All measurements are performed in a laboratory maintained at 23° C.±2° C. and 50%±2% relative humidity.

Analysis is performed on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained and tested as per ISO 1833-1 to quantitatively determine its chemical composition.

### Micro-CT Pore Size Measurement Method

The pore size of a fibrous material composite sample is measured using a micro-CT imaging and analysis method. It is based on analysis of a 3D x-ray sample image obtained on a micro-CT instrument (a suitable instrument is the Scanco μCT 50 available from Scanco Medical AG, Switzerland, or equivalent). The micro-CT instrument is a cone beam micro-tomograph with a shielded cabinet. A maintenance free x-ray tube is used as the source with an adjustable diameter focal spot. The x-ray beam passes through the sample, where some of the x-rays are attenuated by the sample. The extent of attenuation correlates to the mass of material the x-rays have to pass through. The transmitted x-rays continue on to the digital detector array and generate a 2D projection image of the sample. A 3D image of the sample is generated by collecting several individual projection images of the sample as it is rotated, which are then reconstructed into a single 3D image. The instrument is interfaced with a computer running software to control the image acquisition and save the raw data. The 3D image is then analyzed using image analysis software (a suitable image analysis software is MATLAB available from The Mathworks, Inc., Natick, MA, or equivalent) to measure the desired properties of regions within the sample.

### Sample Preparation:

The test sample is prepared either from roll stock of the fibrous material composite, or by removing the fibrous material composite material of interest from an absorbent article. A single layer or one or more sublayers of the fibrous material composite test material is placed onto a rigid, horizontal benchtop and a sharp die cutter is used to punch out a circular sample that has a diameter of 7 mm. The test sample is obtained from an area on the test material that is free of folds or wrinkles, and care is used to prevent any contamination or distortion to the test sample during the preparation process. The body facing side of the test sample should be noted and tracked during the analysis portion in order to identify the discrete layers or sublayers present within the test sample. Additional test samples from separate regions within a given test material can be prepared for analysis and comparison. The test samples are conditioned at about 23° C.±2° C. and about 50%±2% relative humidity for 2 hours prior to testing.

### Image Acquisition:

The micro-CT instrument is set up and calibrated according to the manufacturer's specifications. The test sample is placed into the appropriate holder, between two disks of low-density material, which have a diameter of 7 mm. The test sample is scanned under a compressive load by adding a weight to the uppermost low-density disk, with the mass sufficient to apply a pressure of 2 kPa over the 7 mm diameter test sample. Once the compressive load has been applied, the weight is clamped in place to prevent movement during the scan.

The 3D image field of view is approximately 10 mm on each side in the x-y plane with a resolution of approximately 5124 by 5124 pixel, and with a sufficient number of 2 micron thick slices collected to fully include the entire z-direction of the test sample. The reconstructed 3D image contains isotropic voxel of 2 microns. Images are acquired with a source at 45 kVp and 88 μA with no additional low energy filter. The current and voltage setting should be optimized to produce the maximum contrast in the projection data with sufficient x-ray penetration through the test sample, but once optimized, the settings are held constant for all subsequent test sample replicates. A total of 1800 projection images are obtained with an integration time of 750 ms and 6 averages. The projection images are reconstructed into the 3D image and saved in 16-bit format to preserve the full detector output signal for analysis. A file of the resulting data set is of a proprietary format according to the instrument supplier's instruction, and is referred to as the ISQ file in the following image visualization and analysis steps.

### Image Visualization and Analysis:

The objective of the image analysis is to measure a 3-dimensional void cell diameter in the first layer (or sublayer) and the second layer (or sublayer) of a fibrous material composite test sample. The ISQ files described above, are read into high end image visualization and analysis platform, for example, Avizo 9.2.0 (FEI, Houston, Tex., USA). Upon inspection of obtained visualized 3-dimensional data, 3 different regions in each of the two layers (or sublayers), are analyzed and compared. For each fibrous structure sample there is therefore 6 sub-volumes chosen for measurements of 3-dimensional void size diameter. To make measurements of Porosity and 3D void cell size distribution, the following steps are performed:
1. An automated thresholding algorithm practicing Otsu's method (A Threshold Selection Method from Gray-Level Histograms”, Nobuyuki Otsu, 2EEE Transactions On Systems Man, and Cybernetics, VOL. SMC-9, NO. 1, January 1979) was applied to each of the datasets resulting in a binary image (0-1) representing the fibers (1) and void space (0).
2. A void cell diameter is measured according to the method disclosed in a paper published by Tor Hildebrand (T. Hildebrand and P. Ruegsegger, “A new method for the model-independent assessment of thickness in three-dimensional images. Journal of Microscopy, 185:67-75, 1996). First, the void space is then fitted with spheres of different sizes, where larger spheres cover up smaller spheres using a software working the method disclosed in the paper, for example, IPL software from Scanco Medical, Zurich, Switzerland). This final tessellation of the void space provides a distribution of spheres that completely cover the void space. The volume weighted mean diameter represents the mean void cell diameter. This is implemented through an image analysis platform, for example Matlab R2016B, (Natick, Mass., USA) as module in Avizo 9.2.0.
3. The resulting measurements are brought into Excel 2013. The values of volume weighted mean diameter of the three regions of each layer (or sublayer) are then averaged to produce a single value void cell diameter for that layer (or sublayer). The void cell diameter of the region is a mean pore size of the layer and is reported as Mean Pore Size to the nearest micron.

### Gap SEM Imaging Method

A Scanning Electron Microscope (SEM) is used to obtain images of the cross-section of a test specimen to enable visualization of the microstructure of a fibrous material composite obtained from an absorbent article, specifically the presence or absence of gaps between discrete layers or sublayers within said composite. Quantitative measures of the space between the discrete layers or sublayers are made using image analysis and these measures are then used to determine the presence or absence of a gap between the layers. For the purposes herein, a gap is defined as a space that occurs between fibrous material layers or sublayers of a cross-sectioned test specimen of a fibrous composite material, and said gap will have a height of at least 80 microns and a width of at least 100 microns.

Secondary Electron (SE) images are obtained using an SEM such as the FEI Quanta 450 (available from FEI Company, Hillsboro, OR), or equivalent. The instrument is calibrated according to the manufacturer's instructions prior to use to ensure an accurate distance scale.

Absorbent article samples are conditioned in a laboratory maintained at 23° C.±2° C. and 50%±2% relative humidity for at least 2 hours prior testing. A cross-sectioned test specimen is prepared either from roll stock of the fibrous composite, or by removing the fibrous composite material of interest from an absorbent article. The test sample is taken from a region of the fibrous composite material that excludes all residua of folds or wrinkles. While preparing the test sample and subsequent specimen, care is used to prevent any contamination, stretching, or other distortion to the region that will be analyzed. A cross-sectioned specimen that is at least 12 mm wide and comprises all layers of the fibrous composite material of interest (i.e., the receiving layer) is prepared by sectioning an edge along the width of the test sample with a fresh, new razor blade (such as VWR Single Edge Industrial Razor blade No. 9, surgical carbon steel, or equivalent). To achieve a very clean cut edge, the test specimen can be sectioned while the test sample is submerged in liquid nitrogen. The body-facing side of the test specimen is adhered to a SEM mount using double-sided conductive tape (such as Cu, 3M available from electron microscopy sciences, or equivalent), such that the cross-section can be viewed when the test specimen is tilted backward 90°. In like fashion, a total of five test specimens are prepared, using a fresh new razor blade for each, such that multiple regions within the fibrous composite material can be analyzed.

High resolution SEM images (e.g., 6.8 mega pixel) of the test specimen are obtained as follows. The cross-sectioned surface of the test specimen is initially viewed at a low magnification (e.g., 40×; horizontal field width about 2.5 mm) to identify the presence of gaps between the discrete layers or sublayers within the fibrous composite, and images are acquired. Images of the entire 12 mm wide cross-section are acquired and then subsequently stitched together such that a representation of the entire cross-section can be viewed in a single composite of images. The cross-sectioned surface of the test specimen where there is a gap present is then viewed at a higher magnification (e.g., at least 100×; horizontal field width about 1 mm, and at least 200×; horizontal field width about 300 microns), and images are acquired. The images obtained at each magnification are then subsequently stitched together such that a representation of the entire width of the gap can be viewed in a single composite of images for each magnification.

Quantitative measures are made on the acquired images using image analysis software (e.g., built into the SEM instrument, or standalone software such as ImageJ v. 1.52, National Institute of Health, USA, or equivalent). A suitable image analysis software is used to measure the dimensions of the gaps between the discrete layers within the fibrous composite material. The height of the gap is measured at a minimum of five locations along the width of the gap, and recorded as gap height to the nearest 0.1 micron. The width of the gap is measured as the distance between the first instance where the height of the gap is at least 80 microns, and the last instance where the height of the gap is at least 80 microns, along a contiguous path within the gap. The width is recorded as gap width to the nearest 0.1 micron. If the fibrous composite material does not have at least one gap (height of at least 80 microns that extends for a width of at least 100 microns), the material is determined to be void or free of gaps between discrete layers and/or sublayers.

### Permeability Measurement Method

This method enables calculation of permeability of a material (in Darcys) via measurement of the downward movement of test fluid through a test specimen along the z-direction (plumb direction), over a range of falling hydrohead indicated by decreasing height of a test fluid in a vessel. The decreasing height of the test fluid inside the vessel, as the fluid drains from the bottom of the vessel through the test specimen, is iteratively measured over time during the procedure. From the collected data together with relevant dimensions of portions of the apparatus through which the fluid moves, the measured wet caliper of the test specimen, and constants associated with gravity and properties of the test fluid chosen, flow rate and permeability may be calculated. All measurements are performed in a laboratory maintained at 23° C.±2° C. and 50%±2% relative humidity and test specimens are conditioned in this environment for at least 2 hours prior to testing.

### Apparatus Components

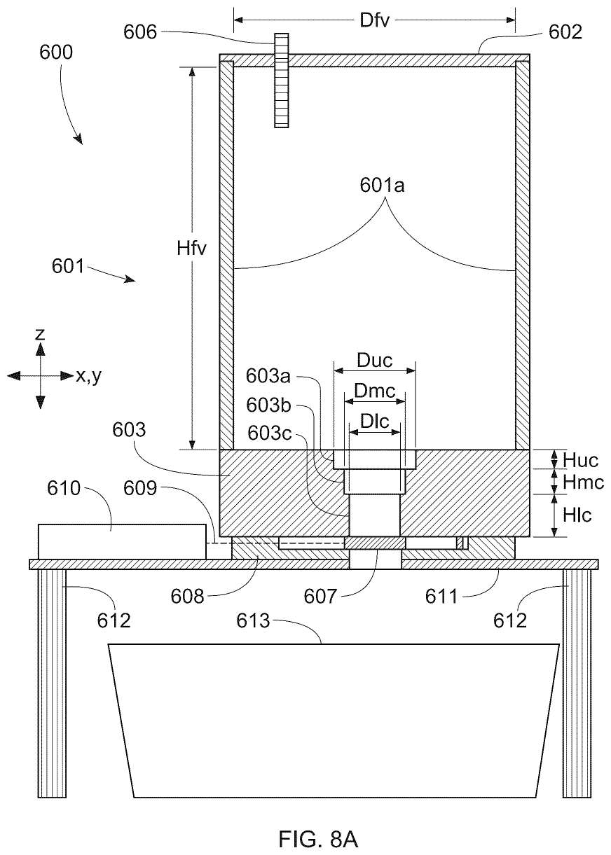

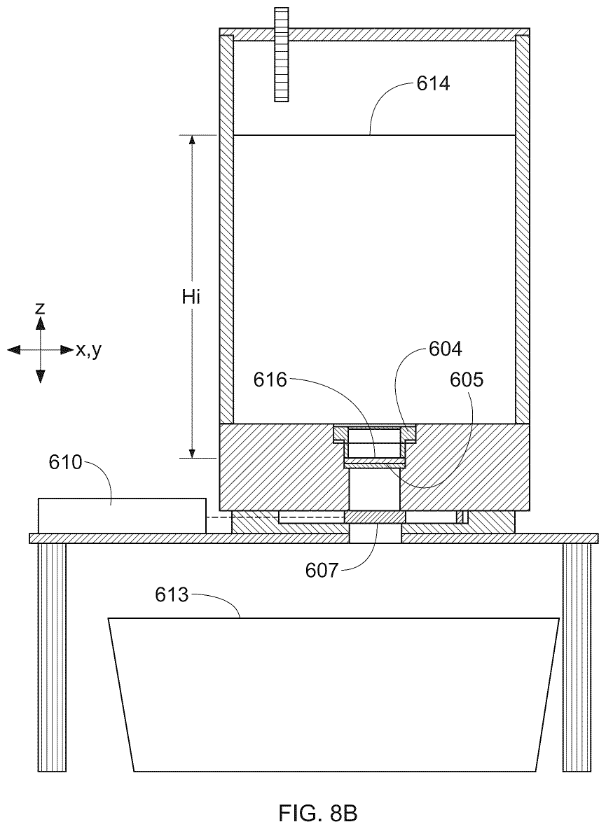

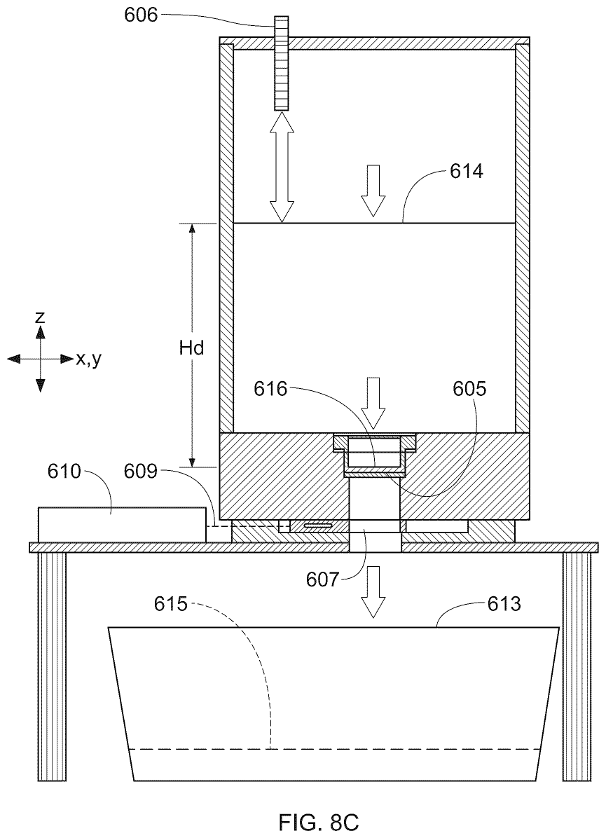

The measurement apparatus 600 and its components are depicted in FIGS. 8A through 10. Referring to FIG. 8A, the apparatus 600 includes a cylindrical fluid vessel 601 including a cylindrical wall 601a with a fitted lid 602 and a base 603 that is sealingly fitted to the bottom of the wall 601a to form fluid vessel 601; a fluid height sensor 606 fitted in and through lid 602; a valve 607 housed in a valve body 608, and a valve actuator 610 mechanically associated with the valve via a linkage 609.

The cylindrical wall has an inside height to the bottom of the lid, Hfv, of 200 mm, an inside diameter of 3⅞ inches (98.425 mm), a wall thickness of ⅜ inch (9.525 mm), and an outer diameter of 4⅝ inches (117.48 mm). The lid 602 is suitably fitted to rest stably on top of the cylindrical wall, but it should not be sealingly fitted thereon; one or more vent holes (not shown) are drilled therethrough to prevent development of negative pressure/vacuum within the fluid vessel as test fluid drains therefrom. The purpose of the lid 602 is to hold and suspend fluid height sensor 606 over the test fluid surface, not to seal the vessel at the top.

Still referring to FIG. 8A, the base 603 has planar, parallel upper and lower surfaces and the upper surface is sealingly affixed to the bottom of the wall 601a. Base 603 is suitably formed or machined to define therewithin a sample chamber having a cylindrical upper chamber portion 603a, a cylindrical middle chamber portion 603b, and a cylindrical lower chamber portion 603c. The three cylindrical chamber portions are coaxial along the vertical/z-direction.

The heights and inner diameters of the three chamber portions are as follows:
Upper chamber portion 603a height Huc: 9.5 mm;
Upper chamber portion 603a inner diameter Duc: 40 mm;
Middle chamber portion 603b height Hmc: 12.5 mm;
Middle chamber portion 603b inner diameter Dmc: 30 mm;
Lower chamber portion 603c height Hlc: 20 mm; and
Lower chamber portion 603a inner diameter Dlc: 26 mm.

Valve body 608 with valve 607 are mounted to the underside of base 603, beneath the lower open end of lower chamber 603c. Valve 607 is configured to be rapidly actuated between fully closed and fully open positions, wherein in the open position, the entirety of lower chamber portion 603c is open to allow fluid to move freely downwardly therefrom without any restriction by valve 607. Valve 607 may be a flat horizontally sliding member, having a circular opening port therethrough, of a diameter of at least 26.0 mm, that is linearly moved to position beneath lower chamber portion 603c upon actuation to the opened position. Alternatively, valve 607 and valve body 608 may have any other suitable configuration adapted to move rapidly between fully closed and fully open positions, wherein when in the fully open position the valve does not present any obstruction to fluid flow downwardly and out from the lower open end of lower chamber portion 603c. Valve 607 and actuator 610 are configured to effect actuation from a fully closed to a fully open position, and vice versa, within no greater than 10 milliseconds for either movement. Actuator 610 may include a solenoid or any other suitable mechanism adapted for this purpose.

Cylindrical wall 601a, lid 602, base 603, and optionally valve body 608 and valve 607, are fabricated of and machined from polished, clear cast acrylic plastic (poly(methyl methacrylate) (PMMA)) stock (known brands include but are not limited to PLEXIGLAS and LUCITE), which may be obtained in various pre-cast tube, rod/bar, disc, sheet and block forms from various suppliers of such materials, such as McMaster-Carr Supply Company (Elmhurst, Illinois). For tube stock used to form wall 601a, tube stock of an inner diameter Dfv varying slightly from that specified herein may be selected, according to availability; in such event, it will be recognized that the corresponding value for the radius r of the fluid vessel, in the equations below, is to be changed to reflect the actual diameter Dfv of the tube stock used.

Fluid height sensor 606 is an ultrasonic height sensor, such as an ML Series part #098-10060, a continuous transmitter through air with an accuracy of about +0.2 mm (TE Connectivity, Schaffhausen, Switzerland and Berwyn, Pennsylvania, USA) or equivalent, interfaced to a computer running software capable of collecting fluid height versus time data throughout the test at a rate of 100 Hz. The fluid height sensor 606 continuously transmits a signal indicating the height of the test fluid within the fluid vessel 601 during the measurement procedure.

The apparatus further includes a support structure, which may include a support platform 611 and height-adjustable legs 612, or any other suitable support structure, configured to stably hold the vessel and valve assembly over a collection vessel 613, with the longitudinal axis of cylindrical wall 601a vertical/plumb and bottom of base 603 level. Where included, a support platform 611 must include an opening or otherwise be configured so as not to obstruct the lower end of lower chamber portion 603c or the fluid exit from valve and valve body 607, 608.

The measurement apparatus further includes a collection vessel 613, of any suitable shape, size and material composition suitable to receive and stably contain the entirety of the volume of test fluid that is used in this method, and fit easily beneath the support structure.

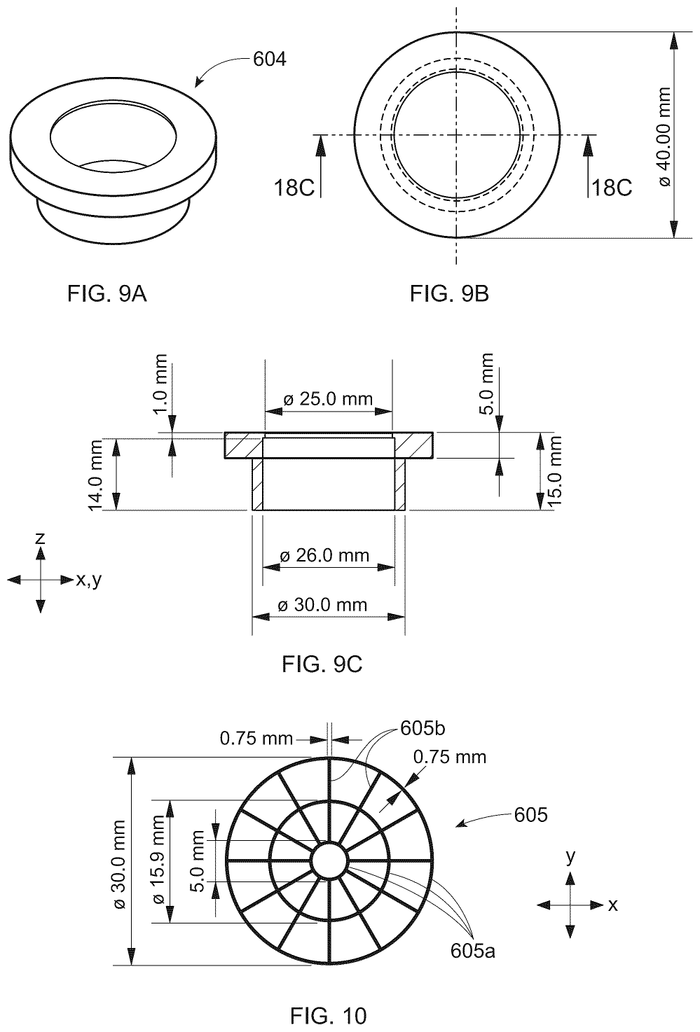

The measurement apparatus further includes a sample weight 604, which is machined of stainless steel to the configuration and dimensions shown in FIGS. 9A-9C. The small radially inwardly-projecting lip at the top portion of sample weight 604 is included for the purpose of providing a feature to grip to facilitate placement and removal of the sample weight 604 into and from the sample chamber.

The measurement apparatus further includes a sample support 605, which has the configuration and dimensions shown in FIG. 10. Sample support 605 has a z-direction caliper of 0.75 mm (which is its height when placed into position within the measurement apparatus in preparation for a measurement procedure). Each of the concentric ring portions 605a and radial spoke portions 605b of sample support 605 shown in FIG. 10 have an x-y plane width of 0.75 mm and a square cross section. Sample support 605 is configured to support a test specimen 616 within middle chamber portion 603b of base 603. Sample support 605 may be cut or machined from any material of suitable strength and corrosion resistance, such as, for example, brass sheet stock.

It will be noted that the outside diameter of sample support 605 and inside diameter of middle chamber portion 603b are both specified above to be 30.0 mm. Sample support 605 is disposed within middle chamber 603b during the measurement procedure. Accordingly, it will be appreciated that either or both of inside diameter of middle chamber portion 603b and outside diameter of sample support 605 may require slight adjustment to provide a small but sufficient clearance to enable sample support 605 to be conveniently inserted into and withdrawn from middle chamber portion 603b.

Similarly, it will be noted that the outside diameter of the lower portion of sample weight 604 and inside diameter of middle chamber portion 603b are both specified above to be 30.0 mm; and the outside diameter of the upper portion of sample weight 604 and inside diameter of upper chamber portion 603a are both specified to be 40.0 mm. The lower portion of sample weight 604 is disposed within middle chamber portion 603b, and the upper portion of sample weight 604 is disposed within upper chamber portion 603a, during the measurement procedure. Accordingly, it will be appreciated that either or both of inside diameter of middle chamber portion 603b and outside diameter of lower portion of sample weight 604, and either or both of inside diameter of upper chamber portion 603a and outside diameter of upper portion sample weight 604, may require slight adjustment to provide a small but sufficient clearance to enable sample weight 605 to be conveniently inserted into and withdrawn from middle chamber portion 603b.

The measurement apparatus further includes a computer (not shown) with suitable software and interfacing equipment configured to communicate with the valve actuator 610 to effect opening and closing of valve 607, and to receive and collect fluid height data from fluid height sensor 606 over time, at a rate of 100 Hz. The person of ordinary skill in the art will have sufficient knowledge and/or resources readily available to obtain components and configure the system including the computer and software to perform the operations described herein.

### Test Fluid Preparation

The test fluid is an aqueous solution of 0.9% w/v saline solution (i.e., 9.0 g of reagent grade NaCl, CAS 7647-14-5, available from any convenient source, diluted to 1 L in deionized water).

Viscosity of the prepared test fluid is performed using a low viscosity rotary viscometer (a suitable instrument is the Cannon LV-2020 Rotary Viscometer with UL adapter, Cannon Instrument Co., State College, Pennsylvania, or equivalent). The appropriate size spindle for the viscosity range is selected, and the instrument is operated and calibrated according to the manufacturer's instructions. Measurements are taken at 23° C. #1° C. and at 30 rpm. Results are reported to the nearest 0.1 centipoise.

### Measurement Procedure

To obtain a test specimen for measurement, lay a single layer of dry subject material out flat on a horizontal work surface, and die-cut a test specimen from it that is circular, with a diameter of 30 mm. Avoid areas of the material having folds, wrinkles or tears when selecting a location for sampling.

If the subject material is a layer component of an absorbent article (e.g., a feminine hygiene pad), for example, a receiving layer or absorbent layer component, obtain a representative sample of the subject material that has not been incorporated into an absorbent article. Alternatively, if only fully manufactured absorbent articles are available as sources of the subject material, from an example thereof, separate the subject layer component from the article without stretching or damaging it. Once the subject layer component has been removed from the article, die-cut out a test specimen as described above. Precondition the test specimen at 23° C.±2° C. and 50%±2% relative humidity for 2 hours prior to testing.

Referring to FIG. 8B, with the fluid valve 607 in closed position, insert the sample support 605 into the middle chamber portion 603b such that it lies horizontal/flat on the lower circumferential lip of middle chamber portion 603b. Using forceps, gently place the test specimen 616 over the sample support 605 so that it lays flat thereon, with no wrinkles. Now gently place the sample weight 604 over/onto the test specimen 616 such that the lower portion of the weight 604 is inserted into middle chamber portion 603b and rests on the test specimen about its circumferential edge, and the upper portion of the weight 604 is nested into the upper chamber portion 603a.

Now slowly add the previously prepared test solution to the fluid vessel 601, until an initial fluid surface 614 height Hi of 150 mm above the upper surface of the test specimen 616 is reached.

Allow the test specimen 616 to equilibrate within the filled sample chamber for about 60 seconds, and ensure there are no bubbles present on the surface of the test fluid or surface of the test specimen. If bubbles are present on the fluid surface, remove or pop them using a clean instrument. If bubbles are present on the upper surface of the test specimen 616, use a clean, round tip lab stirring rod to gently dislodge them, exercising care not to dislodge fibers (if the test specimen is fibrous), or stretch or damage the test specimen.

Secure the fluid height sensor 606 to the lid 602, and then place and fit the lid 602 over cylindrical wall 601a. Adjust the position of the fluid height sensor 606, if necessary, prior to the start of the test so as to prevent it from contacting the starting surface of the test fluid. Initially, the lower tip of the sensor 606 should be about 170 mm from the upper surface of the test specimen 616.

Position the collection vessel 613 below the valve 607.

Referring now to FIG. 8C, to start the measurement, simultaneously open the valve 607 and start the acquisition of decreasing fluid height Hd and time data to the nearest 0.01 mm and 0.01 seconds, respectively, with a data acquisition rate of 100 Hz. Test fluid will flow under gravitational pull through the sample chamber and through test specimen 616, sample support 605 and open valve 607, down into collection vessel 613, and test fluid surface 614 will fall while a surface 615 of collected fluid will rise. Height sensor 606 will sense and transmit data concerning the height of test fluid surface 614 at the designated sensing frequency, over time. The measurement is ended and the valve 607 is closed when test fluid has ceased exiting the valve, or after 1,000 seconds have elapsed, whichever occurs first. Remove the lid 602. Lift the sample weight 604 out of the sample chamber, and, using forceps, gently remove the wet test specimen 616 from the sample chamber, and proceed to measure the wet caliper of the test specimen.

The wet caliper of the test specimen 616 is measured promptly after completion of the measurement procedure, using a manually-operated micrometer equipped with a pressure foot capable of exerting a steady pressure of 2.07 kPa+0.07 kPa. The manually-operated micrometer is a dead-weight type instrument with readings accurate to 0.01 mm. A suitable instrument is Mitutoyo Series 543 ID-C Digimatic, available from Avantor/VWR International (Radnor, Pennsylvania) or equivalent. The pressure foot is a flat circular moveable face with a diameter of 19 mm. The test specimen is supported by a horizontal flat reference platform that is larger than and parallel to the surface of the pressure foot. Zero the micrometer against the horizontal flat reference platform. Transfer the wet test specimen 616 to the reference platform of the micrometer such that the specimen 616 is centered and lies horizontally and flat beneath the pressure foot. The pressure foot is lowered by hand with a descent rate of 3+1 mm/s until the full pressure (2.07 kPa) is applied to the test specimen. After 5 seconds elapse, the caliper of the wet test specimen is recorded as specimen caliper, to the nearest 0.01 mm. The test specimen is then discarded.

Remove test fluid inside the fluid vessel 601 and sample chamber, if any remains therein.

The procedure is repeated for a total of three replicate test specimens.

A separate “blank” run measurement is performed by following the procedure described above, but with only the sample support 605 and sample weight 604 present in the sample chamber (i.e., no test specimen is present). Note that the initial test fluid height Hi will be 150 mm above the upper surface of the sample support 605, rather than a surface of a specimen. This blank measurement will enable the permeability of the sample support 605 to be considered, when calculating the permeability of the test specimen.

### Permeability Calculation

Total permeability, kₜₒₜₐₗ, is the permeability of the test specimen plus the sample support, calculated from the time and volume of flow through a fluid height decrease from 150 mm test fluid to 130 mm test fluid. Total permeability is calculated for each replicate test specimen using the following equation, and recorded to the nearest 0.01 E⁻¹⁰ m²:

$$ln ( \frac{Hi}{Hd} ) = \frac{{k}_{total} ρ g}{μ {L}_{total}} * \frac{{R}^{2}}{{r}^{2}} * t$$

thus, solved for kₜₒₜₐₗ:

$${k}_{total} = \frac{[ ( \frac{ln ( \frac{Hi}{Hd} )}{t} ) * \frac{{r}^{2}}{{R}^{2}} * μ * {L}_{total} ]}{ρ g} ;$$

where:
Hi=initial test fluid height (150 mm)
Hd=test fluid height as decreased at time t (for the present calculation, this is 130 mm)
t=time (seconds) elapsed when fluid height has decreased to 130 mm
kₜₒₜₐₗ=combined permeability of the test specimen and the sample support
ρ=density of the test fluid (kg/m³)
g=gravitational constant (9.81 m/s²)
μ=viscosity of the test fluid (assumed to be 0.00109 kg/m-s)
Lₜₒₜₐₗ=combined caliper of the wet test specimen and the sample support (m)
R=the radius of the surface area of the test specimen through which the fluid flows ((26 mm/2)×(1m/1,000 mm)=0.013 m)
r=radius of the inside of the fluid vessel ((98.425 mm/2)×(1 m/1,000 mm)=0.049213 m)

The permeability of the sample support 605, kₛₛᵤₚ, is calculated in a similar manner, from the time and volume of flow through a fluid height decrease from 150 mm test fluid to 130 mm test fluid in the “blank” run. The permeability of the sample support 605 alone is described by the following equation, and recorded to the nearest 0.01 E⁻¹⁰ m²:

$$ln ( \frac{Hi}{Hd} ) = \frac{{k}_{ssup} ρ g}{μ {L}_{ssup}} * \frac{{R}^{2}}{{r}^{2}} * t$$

thus, solved for kₛₛᵤₚ:

$${k}_{ssup} = \frac{[ ( \frac{ln ( \frac{Hi}{Hd} )}{t} ) * \frac{{r}^{2}}{{R}^{2}} * μ * {L}_{frit} ]}{ρ g} ;$$

where:

$${L}_{ssup} = the caliper of the sample support 605 ( 0.00075 m )$$

The permeability of each replicate test specimen, k_{specimen}, is calculated from the following equation, then multiplied by 1.01324998 E⁺¹² and recorded to the nearest 0.1 Darcy:

$${k}_{specimen} = \frac{{L}_{specimen}}{( \frac{{L}_{total}}{{k}_{total}} ) - ( \frac{{L}_{ssup}}{{k}_{ssup}} )}$$

Now calculate the arithmetic mean of the test specimen permeability, k_{specimen}, across all three replicate test specimens, and report as Through Plane Permeability to the nearest 0.1 Darcy.

### Combinations

Paragraph 1. An absorbent article comprising: a receiving layer, a backsheet, and an absorbent core disposed between the receiving layer and the backsheet, the receiving layer comprising a multi-sublayer structure, the multi-sublayer structure comprising a first sublayer and a second sublayer, wherein the first sublayer is disposed adjacent to a wearer's skin, wherein the first sublayer has a basis weight of from about 15 gsm to about 45 gsm, wherein the second sublayer is located below the first sublayer and between the first sublayer and the absorbent core, wherein the second sublayer comprises nonwoven fibers having a basis weight of from about 40 gsm to about 75 gsm, the nonwoven fibers comprising from about 15 to about 35 weight percent of cellulosic fibers, from about 65 to about 85 weight percent of bonding fibers, wherein the cellulosic fibers and the bonding fibers have a decitex of less than about 2, wherein the first sublayer is integrated with the second sublayer, and wherein the integration comprises micro-fiber integration and large-fiber integration.

Paragraph 2. The absorbent article according to paragraph 1, wherein the absorbent article is free of adhesive between the first sublayer and the second sublayer.

Paragraph 3. The absorbent article according to any of the preceding paragraphs, wherein the receiving layer comprises a plurality of large-fiber integration areas, wherein the large-fiber integration areas are formed by needle punching the receiving layer and configured to transfer fluid from a wearer-facing surface to a garment-facing surface of the receiving layer.

Paragraph 4. The absorbent article according to paragraph 3, wherein there are no gaps in between the first sublayer and the second sublayer at each of the plurality of large-fiber integration areas.

Paragraph 5. The absorbent article according to any of the preceding paragraphs, wherein the receiving layer is free of gaps having a gap height of at least 80 microns and a gap width of at least 100 microns.

Paragraph 6. The absorbent article according to any of the preceding paragraphs, comprising large-fiber integration with a density of between 90 and 220 integration sites per square centimeter.

Paragraph 7. The absorbent article according to any of the preceding paragraphs, wherein the second layer further comprises from about 35 to about 55 weight percent divider fibers having a decitex of from about 0.5 to about 2.

Paragraph 8. The absorbent article according to paragraph 7, wherein the divider fibers are non-cylindrical polypropylene.

Paragraph 9. The absorbent article according to any of the preceding paragraphs, wherein the second layer has a caliper factor of from about 0.25 mm to about 0.35 mm.

Paragraph 10. The absorbent article according to any of the preceding paragraphs, wherein the first sublayer and the second sublayer are carded nonwovens.

Paragraph 11. The absorbent article of according to paragraph 10, wherein the first sublayer is made from nonwoven fibers having an average denier of from 3.0 to 5.0.

Paragraph 12. The absorbent article according to paragraph 11, wherein the first layer fibers comprise fibers having a sheath-core bicomponent configuration.

Paragraph 13. The absorbent article according to paragraph 12, wherein the sheath component comprises polyethylene and the core component comprises polyethylene terephthalate.

Paragraph 14. The absorbent article according to paragraph 13, wherein a weight ratio of the polyethylene fibers and the polyethylene terephthalate fibers is 40:60 to 60:40.

Paragraph 15. The absorbent article according to any of the preceding paragraphs, wherein the first sublayer comprises a blend of intermixed hydrophilic fibers and hydrophobic fibers.

Paragraph 16. The absorbent article according to paragraph 15, wherein the first sublayer comprises a greater weight percentage of hydrophilic fibers than hydrophobic fibers.

Paragraph 17. The absorbent article according to any of the preceding paragraphs, wherein a portion of the backsheet is joined to a portion of the receiving layer.

Paragraph 18. The absorbent article according to any of the preceding paragraphs, comprising a wing layer joined to at least one of the receiving layer and the backsheet, wherein the wing layer extends away from a first longitudinal edge of the receiving layer to form a wing.

Paragraph 19. The absorbent article according to paragraph 18, comprising a second wing layer joined to at least one of the receiving layer and the backsheet, wherein the second wing layer extends from a second longitudinal edge of the receiving layer to from a second wing.

Paragraph 20. The absorbent article according to paragraph 19, wherein the wing and the second wing are asymmetrical about a longitudinal axis of the absorbent article.

Paragraph 21. An absorbent article comprising: a receiving layer, a backsheet, and an absorbent core disposed between the receiving layer and the backsheet, the receiving layer comprising a multi-sublayer structure, the multi-sublayer structure comprising a first sublayer and a second sublayer, wherein the first sublayer is disposed adjacent to a wearer's skin, wherein the first sublayer has a basis weight of from about 15 gsm to about 45 gsm, wherein the second sublayer is located below the first sublayer and between the first sublayer and the absorbent core, wherein the second sublayer comprises nonwoven fibers having a basis weight of from about 40 gsm to about 75 gsm, the nonwoven fibers comprising from about 15 to about 35 weight percent of cellulosic fibers, from about 65 to about 85 weight percent of bonding fibers, wherein the cellulosic fibers and the bonding fibers have a decitex of less than about 2, wherein the first sublayer is integrated with the second sublayer, and wherein the integration comprises a plurality of large-fiber integration areas, and wherein the first sublayer has a first mean pore size and the second sublayer has a second mean pore size, and the second mean pore size is at least about 40% of the first mean pore size, as determined by the Micro-CT Pore Size Measurement Method.

Paragraph 22. The absorbent article of claim 21, wherein the first mean pore size is from about 60% to about 90% of the second mean pore size, as determined by the Micro-CT Pore Size Measurement Method.

Paragraph 23. The absorbent article according to paragraph 21, wherein the absorbent article is free of adhesive between the first sublayer and the second sublayer.

Paragraph 24. The absorbent article according to any of the preceding paragraphs, wherein the receiving layer comprises a plurality of large-fiber integration areas, wherein the large-fiber integration areas are formed by needle punching the receiving layer and configured to transfer fluid from a wearer-facing surface to a garment-facing surface of the receiving layer.

Paragraph 25. The absorbent article according to paragraph 24, wherein there are no gaps in between the first sublayer and the second sublayer at each of the plurality of large-fiber integration areas.

Paragraph 26. The absorbent article according to any of the preceding paragraphs, wherein the receiving layer is free of gaps having a gap height of at least 80 microns and a gap width of at least 100 microns.

Paragraph 27. The absorbent article according to any of the preceding paragraphs, comprising large-fiber integration with a density of between 90 and 220 integration sites per square centimeter.

Paragraph 28. The absorbent article according to any of the preceding paragraphs, wherein the second layer further comprises from about 35 to about 55 weight percent divider fibers having a decitex of from about 0.5 to about 2.

Paragraph 29. The absorbent article according to paragraph 28, wherein the divider fibers are non-cylindrical polypropylene.

Paragraph 30. The absorbent article according to any of the preceding paragraphs, wherein the second layer has a caliper factor of from about 0.25 mm to about 0.35 mm.

Paragraph 31. The absorbent article according to any of the preceding paragraphs, wherein the first sublayer and the second sublayer are carded nonwovens.

Paragraph 32. The absorbent article of according to paragraph 31, wherein the first sublayer is made from nonwoven fibers having an average denier of from 3.0 to 5.0.

Paragraph 33. The absorbent article according to paragraph 32, wherein the first layer fibers comprise fibers having a sheath-core bicomponent configuration.

Paragraph 34. The absorbent article according to paragraph 33, wherein the sheath component comprises polyethylene and the core component comprises polyethylene terephthalate.

Paragraph 35. The absorbent article according to paragraph 34, wherein a weight ratio of the polyethylene fibers and the polyethylene terephthalate fibers is 40:60 to 60:40.

Paragraph 36. The absorbent article according to any of the preceding paragraphs, wherein the first sublayer comprises a blend of intermixed hydrophilic fibers and hydrophobic fibers.

Paragraph 37. The absorbent article according to paragraph 36, wherein the first sublayer comprises a greater weight percentage of hydrophilic fibers than hydrophobic fibers.

Paragraph 38. The absorbent article according to any of the preceding paragraphs, wherein a portion of the backsheet is joined to a portion of the receiving layer.

Paragraph 39. The absorbent article according to any of the preceding paragraphs, comprising a wing layer joined to at least one of the receiving layer and the backsheet, wherein the wing layer extends away from a first longitudinal edge of the receiving layer to form a wing.

Paragraph 40. The absorbent article according to paragraph 39, comprising a second wing layer joined to at least one of the receiving layer and the backsheet, wherein the second wing layer extends from a second longitudinal edge of the receiving layer to from a second wing.

Paragraph 41. The absorbent article according to paragraph 40, wherein the wing and the second wing are asymmetrical about a longitudinal axis of the absorbent article.

Paragraph 42. An absorbent article comprising: a receiving layer, a backsheet, and an absorbent core disposed between the receiving layer and the backsheet, the receiving layer comprising a multi-sublayer structure, the multi-sublayer structure comprising a first sublayer and a second sublayer, wherein the first sublayer is disposed adjacent to a wearer's skin, wherein the first sublayer has a basis weight of from about 15 gsm to about 45 gsm, wherein the second sublayer is located below the first sublayer and between the first sublayer and the absorbent core, wherein the second sublayer comprises nonwoven fibers having a basis weight of from about 40 gsm to about 75 gsm, the nonwoven fibers comprising from about 15 to about 35 weight percent of cellulosic fibers, from about 65 to about 85 weight percent of bonding fibers, wherein the cellulosic fibers and the bonding fibers have a decitex of less than about 2, wherein the first sublayer is integrated with the second sublayer, and wherein the integration comprises a plurality of large-fiber integration areas, and wherein the first sublayer has a first through plane permeability and the second sublayer has a second through plane permeability, and wherein the second through plane permeability is at least about 30% of the first through plane permeability.

Paragraph 43. The absorbent article of claim 42, wherein the second through plane permeability is from about 30% to about 70% of the first through plane permeability.

Paragraph 44. The absorbent article according to paragraph 42, wherein the absorbent article is free of adhesive between the first sublayer and the second sublayer.

Paragraph 45. The absorbent article according to any of the preceding paragraphs, wherein the receiving layer comprises a plurality of large-fiber integration areas, wherein the large-fiber integration areas are formed by needle punching the receiving layer and configured to transfer fluid from a wearer-facing surface to a garment-facing surface of the receiving layer.

Paragraph 46. The absorbent article according to paragraph 45, wherein there are no gaps in between the first sublayer and the second sublayer at each of the plurality of large-fiber integration areas.

Paragraph 47. The absorbent article according to any of the preceding paragraphs, wherein the receiving layer is free of gaps having a gap height of at least 80 microns and a gap width of at least 100 microns.

Paragraph 48. The absorbent article according to any of the preceding paragraphs, comprising large-fiber integration with a density of between 90 and 220 integration sites per square centimeter.

Paragraph 49. The absorbent article according to any of the preceding paragraphs, wherein the second layer further comprises from about 35 to about 55 weight percent divider fibers having a decitex of from about 0.5 to about 2.

Paragraph 50. The absorbent article according to paragraph 49, wherein the divider fibers are non-cylindrical polypropylene.

Paragraph 51. The absorbent article according to any of the preceding paragraphs, wherein the second layer has a caliper factor of from about 0.25 mm to about 0.35 mm.

Paragraph 52. The absorbent article according to any of the preceding paragraphs, wherein the first sublayer and the second sublayer are carded nonwovens.

Paragraph 53. The absorbent article of according to paragraph 52, wherein the first sublayer is made from nonwoven fibers having an average denier of from 3.0 to 5.0.

Paragraph 54. The absorbent article according to paragraph 53, wherein the first layer fibers comprise fibers having a sheath-core bicomponent configuration.

Paragraph 55. The absorbent article according to paragraph 54, wherein the sheath component comprises polyethylene and the core component comprises polyethylene terephthalate.

Paragraph 56. The absorbent article according to paragraph 55, wherein a weight ratio of the polyethylene fibers and the polyethylene terephthalate fibers is 40:60 to 60:40.

Paragraph 57. The absorbent article according to any of the preceding paragraphs, wherein the first sublayer comprises a blend of intermixed hydrophilic fibers and hydrophobic fibers.

Paragraph 58. The absorbent article according to paragraph 57, wherein the first sublayer comprises a greater weight percentage of hydrophilic fibers than hydrophobic fibers.

Paragraph 59. The absorbent article according to any of the preceding paragraphs, wherein a portion of the backsheet is joined to a portion of the receiving layer.

Paragraph 60. The absorbent article according to any of the preceding paragraphs, comprising a wing layer joined to at least one of the receiving layer and the backsheet, wherein the wing layer extends away from a first longitudinal edge of the receiving layer to form a wing.

Paragraph 61. The absorbent article according to paragraph 60, comprising a second wing layer joined to at least one of the receiving layer and the backsheet, wherein the second wing layer extends from a second longitudinal edge of the receiving layer to from a second wing.

Paragraph 62. The absorbent article according to paragraph 61, wherein the wing and the second wing are asymmetrical about a longitudinal axis of the absorbent article.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a receiving layer, a backsheet, and an absorbent core disposed between the receiving layer and the backsheet, the receiving layer comprising a multi-sublayer structure,
the multi-sublayer structure comprising a first sublayer and a second sublayer,
wherein the first sublayer is disposed adjacent to a wearer's skin, wherein the first sublayer has a basis weight of from about 15 gsm to about 45 gsm,
wherein the second sublayer is located below the first sublayer and between the first sublayer and the absorbent core, wherein the second sublayer comprises nonwoven fibers having a basis weight of from about 40 gsm to about 75 gsm, the nonwoven fibers comprising from about 15 to about 35 weight percent of cellulosic fibers, from about 65 to about 85 weight percent of bonding fibers, wherein the cellulosic fibers and the bonding fibers have a decitex of less than about 2,
wherein the first sublayer is integrated with the second sublayer, and wherein the integration comprises micro-fiber integration and large-fiber integration.

2. The absorbent article of claim 1, wherein the absorbent article is free of adhesive between the first sublayer and the second sublayer.

3. The absorbent article of claim 1, wherein the receiving layer comprises a plurality of large-fiber integration areas, wherein the large-fiber integration areas are formed by needle punching the receiving layer and configured to transfer fluid from a wearer-facing surface to a garment-facing surface of the receiving layer.

4. The absorbent article of claim 3, wherein there are no gaps between the first sublayer and the second sublayer at each of the plurality of large-fiber integration areas.

5. The absorbent article of claim 1, wherein the receiving layer is free of gaps having a gap height of at least 80 microns and a gap width of at least 100 microns.

6. The absorbent article of claim 1, comprising large-fiber integration with a density of between 90 and 220 integration sites per square centimeter.

7. The absorbent article of claim 1, wherein the second layer further comprises from about 35 to about 55 weight percent divider fibers having a decitex of from about 0.5 to about 2.

8. The absorbent article of claim 7, wherein the divider fibers are non-cylindrical polypropylene.

9. The absorbent article of claim 1, wherein the second layer has a caliper factor of from about 0.25 mm to about 0.35 mm.

10. The absorbent article of claim 1, wherein the first sublayer and the second sublayer are carded nonwovens.

11. The absorbent article of claim 10, wherein the first sublayer is made from nonwoven fibers having an average denier of from 3.0 to 5.0.

12. The absorbent article of claim 11, wherein the first layer fibers comprises fibers having a sheath-core bicomponent configuration.

13. The absorbent article of claim 12, wherein the sheath component comprises polyethylene and the core component comprises polyethylene terephthalate.

14. The absorbent article of claim 13, wherein a weight ratio of the polyethylene fibers and the polyethylene terephthalate fibers is 40:60 to 60:40.

15. The absorbent article of claim 1, wherein the first sublayer comprises a blend of intermixed hydrophilic fibers and hydrophobic fibers.

16. The absorbent article of claim 15, wherein the first sublayer comprises a greater weight percentage of hydrophilic fibers than hydrophobic fibers.

17. The absorbent article of claim 1, wherein a portion of the backsheet is joined to a portion of the receiving layer.

18. The absorbent article of claim 1, comprising a wing layer joined to at least one of the receiving layer and the backsheet, wherein the wing layer extends away from a first longitudinal edge of the receiving layer to form a wing.

19. An absorbent article comprising:
a receiving layer, a backsheet, and an absorbent core disposed between the receiving layer and the backsheet, the receiving layer comprising a multi-sublayer structure,
the multi-sublayer structure comprising a first sublayer and a second sublayer,
wherein the first sublayer is disposed adjacent to a wearer's skin, wherein the first sublayer has a basis weight of from about 15 gsm to about 45 gsm,
wherein the second sublayer is located below the first sublayer and between the first sublayer and the absorbent core, wherein the second sublayer comprises nonwoven fibers having a basis weight of from about 40 gsm to about 75 gsm, the nonwoven fibers comprising from about 15 to about 35 weight percent of cellulosic fibers, from about 65 to about 85 weight percent of bonding fibers, wherein the cellulosic fibers and the bonding fibers have a decitex of less than about 2,
wherein the first sublayer is integrated with the second sublayer, and wherein the integration comprises a plurality of large-fiber integration areas, and
wherein the first sublayer has a first mean pore size and the second sublayer has a second mean pore size, and the second mean pore size is at least about 40% of the first mean pore size, as determined by the Micro-CT Pore Size Measurement Method.

20. An absorbent article comprising:
a receiving layer, a backsheet, and an absorbent core disposed between the receiving layer and the backsheet, the receiving layer comprising a multi-sublayer structure,
the multi-sublayer structure comprising a first sublayer and a second sublayer,
wherein the first sublayer is disposed adjacent to a wearer's skin, wherein the first sublayer has a basis weight of from about 15 gsm to about 45 gsm,
wherein the second sublayer is located below the first sublayer and between the first sublayer and the absorbent core, wherein the second sublayer comprises nonwoven fibers having a basis weight of from about 40 gsm to about 75 gsm, the nonwoven fibers comprising from about 15 to about 35 weight percent of cellulosic fibers, from about 65 to about 85 weight percent of bonding fibers, wherein the cellulosic fibers and the bonding fibers have a decitex of less than about 2,
wherein the first sublayer is integrated with the second sublayer, and wherein the integration comprises a plurality of large-fiber integration areas, and
wherein the first sublayer has a first through plane permeability and the second sublayer has a second through plane permeability, and wherein the second through plane permeability is at least about 30% of the first through plane permeability.
